SEQUENCE LISTING (28 sequences)

SEQ ID NO: 1 - DNA, 762 bp, Artificial Sequence
  Human IgG1 Fc region containing mutations
atgagatttccttcaatttttactgctgttttattcgcagcatcctccgcattagctgctgaaccaaagtcttgtgacaagacacacacttgtccaccatgtccagctccagaattgttgggtggtccatccgtttttttggctccaccaaagccaaaggacactttgtacatcactagagaaccagaggttacatgtgttgttgttgacgtttctcacgaggacccagaggttaagttcaactggtacgttgacggtgttgaagttcacaacgctaagactaagccaagagaagagcagtacaactccacttacagagttgtttccgttttgactgttttgcaccaggattggttgaacggtaaagaatacaagtgtaaggtttccaacaaggctttgccagctccaatcgaaaagactatctccaaggctaagggtcaaccaagagagccacaggtttacactttgccaccatccagagatgagttgactaagaaccaggtttccttgacttgtttggttaagggattctacccatccgacattgctgttgagtgggaatctaacggtcaaccagagaacaactacaagactactccaccagttttggattctgacggttccttcttcttgtactccaagttgactgttgacaagtccagatggcaacagggtaacgttttctcctgttccgttatgcatgaggctttgaagtttcactacactcaaaagtccttgtctttgtcccctggtaagtaatga

SEQ ID NO: 2 - protein, 252 aa, Artificial Sequence
  Human IgG1 Fc region containing mutations
MRFPSIFTAVLFAASSALAAEPKSCDKTHTCPPCPAPELLGGPSVFLAPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 3 - DNA, 763 bp, Artificial Sequence
  Human IgG1 region containing mutations
atgagatttccttcaatttttactgctgttttattcgcagcatcctccgcattagctgctgaaccaaagtcttgtgacaagacacacacttgtccaccatgtccagctccagaattgttgggtggtccatccgtttttttgtacccaccaaagccaaaggacactttgtacatcactagagaaccagaggttacatgtgttgttggtgacgtttctcacgaggacccagaggttaagttcaactggtacgttgacggtgttgaagttcacaacgctaagactaagccaagagaagagcagtacaactccacttacagagttgtttccgttttgactgttttgcaccaggattggttgaacggtaaagaatacaagtgtaaggtttccaacaaggctttgccagctccaatcgaaaagactatctccaaggctaagggtcaaccaagagagccacaggtttacactttgccaccatccagagatgagttgactaagaaccaggtttccttgacttgtttggttaagggattctacccatccgacattgctgttgagtgggaatctaacggtcaaccagagaacaactacaagactactccaccagttttggattctgacggttccttcttcttgtactccaagttgactgttgacaagtccagatggcaacagggtaacgttttctcctgttccgttatgcatgaggctttgaagtttcactacactcaaaagtccttgtctttgtcccctggtaagtaatgag

SEQ ID NO: 4 - protein, 252 aa, Artificial Sequence
  Human IgG1 Fc region containing mutations
MRFPSIFTAVLFAASSALAAEPKSCDKTHTCPPCPAPELLGGPSVFLYPPKPKDTLYITREPEVTCVVGDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 5 - DNA, 770 bp, Artificial Sequence
  Human IgG1 Fc region containing mutations
atgagatttccttcaatttttactgctgttttattcgcagcatcctccgcattagctgctgaaccaaagtcttgtgacaagacacacacttgtccaccatgtccagctccagaattgttgggtggtccatccgtttttttgttgccaccaaagccaaaggacactttgtacatcactagagaaccagaggttacatgtgttgttaacgacgtttctcacgaggacccagaggttaagttcaactggtacgttgacggtgttgaagttcacaacgctaagactaagccaagagaagagcagtacaactccacttacagagttgtttccgttttgactgttttgcaccaggattggttgaacggtaaagaatacaagtgtaaggtttccaacaaggctttgccagctccaatcgaaaagactatctccaaggctaagggtcaaccaagagagccacaggtttacactttgccaccatccagagatgagttgactaagaaccaggtttccttgacttgtttggttaagggattctacccatccgacattgctgttgagtgggaatctaacggtcaaccagagaacaactacaagactactccaccagttttggattctgacggttccttcttcttgtactccaagttgactgttgacaagtccagatggcaacagggtaacgttttctcctgttccgttatgcatgaggctttgaagtttcactacactcaaaagtccttgtctttgtcccctggtaagtaatgaggccggcc

SEQ ID NO: 6 - protein, 252 aa, Artificial Sequence
  Human IgG1 region containing mutations
MRFPSIFTAVLFAASSALAAEPKSCDKTHTCPPCPAPELLGGPSVFLLPPKPKDTLYITREPEVTCVVNDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 7 - DNA, 762 bp, Artificial Sequence
  Human IgG1 Fc region containing mutations
atgagatttccttcaatttttactgctgttttattcgcagcatcctccgcattagctgctgaaccaaagtcttgtgacaagacacacacttgtccaccatgtccagctccagaattgttgggtggtccatccgtttttttgttgccaccaaagccaaaggacactttgtacatcactagagaaccagaggttacatgtgttgttgctgacgtttctcacgaggacccagaggttaagttcaactggtacgttgacggtgttgaagttcacaacgctaagactaagccaagagaagagcagtacaactccacttacagagttgtttccgttttgactgttttgcaccaggattggttgaacggtaaagaatacaagtgtaaggtttccaacaaggctttgccagctccaatcgaaaagactatctccaaggctaagggtcaaccaagagagccacaggtttacactttgccaccatccagagatgagttgactaagaaccaggtttccttgacttgtttggttaagggattctacccatccgacattgctgttgagtgggaatctaacggtcaaccagagaacaactacaagactactccaccagttttggattctgacggttccttcttcttgtactccaagttgactgttgacaagtccagatggcaacagggtaacgttttctcctgttccgttatgcatgaggctttgaagtttcactacactcaaaagtccttgtctttgtcccctggtaagtaatga

SEQ ID NO: 8 - protein, 252 aa, Artificial Sequence
  Human IgG1 Fc region containing mutations
MRFPSIFTAVLFAASSALAAEPKSCDKTHTCPPCPAPELLGGPSVFLLPPKPKDTLYITREPEVTCVVADVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 9 - DNA, 762 bp, Artificial Sequence
  Human IgG1 Fc region containing mutations
atgagatttccttcaatttttactgctgttttattcgcagcatcctccgcattagctgctgaaccaaagtcttgtgacaagacacacacttgtccaccatgtccagctccagaattgttgggtggtccatccgtttttttggttccaccaaagccaaaggacactttgtacatcactagagaaccagaggttacatgtgttgttggtgacgtttctcacgaggacccagaggttaagttcaactggtacgttgacggtgttgaagttcacaacgctaagactaagccaagagaagagcagtacaactccacttacagagttgtttccgttttgactgttttgcaccaggattggttgaacggtaaagaatacaagtgtaaggtttccaacaaggctttgccagctccaatcgaaaagactatctccaaggctaagggtcaaccaagagagccacaggtttacactttgccaccatccagagatgagttgactaagaaccaggtttccttgacttgtttggttaagggattctacccatccgacattgctgttgagtgggaatctaacggtcaaccagagaacaactacaagactactccaccagttttggattctgacggttccttcttcttgtactccaagttgactgttgacaagtccagatggcaacagggtaacgttttctcctgttccgttatgcatgaggctttgaagtttcactacactcaaaagtccttgtctttgtcccctggtaagtaatga

SEQ ID NO: 10 - protein, 252 aa, Artificial Sequence
  Human IgG1 Fc region containing mutations
MRFPSIFTAVLFAASSALAAEPKSCDKTHTCPPCPAPELLGGPSVFLVPPKPKDTLYITREPEVTCVVGDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 11 - DNA, 762 bp, Artificial Sequence
  Human IgG1 Fc region containing mutations
atgagatttccttcaatttttactgctgttttattcgcagcatcctccgcattagctgctgaaccaaagtcttgtgacaagacacacacttgtccaccatgtccagctccagaattgttgggtggtccatccgtttttttggctccaccaaagccaaaggacactttgtacatcactagagaaccagaggttacatgtgttgttgttgctgtttctcacgaggacccagaggttaagttcaactggtacgttgacggtgttgaagttcacaacgctaagactaagccaagagaagagcagtacaactccacttacagagttgtttccgttttgactgttttgcaccaggattggttgaacggtaaagaatacaagtgtaaggtttccaacaaggctttgccagctccaatcgaaaagactatctccaaggctaagggtcaaccaagagagccacaggtttacactttgccaccatccagagatgagttgactaagaaccaggtttccttgacttgtttggttaagggattctacccatccgacattgctgttgagtgggaatctaacggtcaaccagagaacaactacaagactactccaccagttttggattctgacggttccttcttcttgtactccaagttgactgttgacaagtccagatggcaacagggtaacgttttctcctgttccgttatgcatgaggctttgaagtttcactacactcaaaagtccttgtctttgtcccctggtaagtaatga

SEQ ID NO: 12 - protein, 252 aa, Artificial Sequence
  Human IgG1 region containing mutations
MRFPSIFTAVLFAASSALAAEPKSCDKTHTCPPCPAPELLGGPSVFLAPPKPKDTLYITREPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 13 - DNA, 762 bp, Artificial Sequence
  Human IgG1 Fc region containing mutations
atgagatttccttcaatttttactgctgttttattcgcagcatcctccgcattagctgctgaaccaaagtcttgtgacaagacacacacttgtccaccatgtccagctccagaattgttgggtggtccatccgtttttttgtttccaccaaagccaaaggacactttgtacatcactagagaaccagaggttacatgtgttgttgccgctgtttctcacgaggacccagaggttaagttcaactggtacgttgacggtgttgaagttcacaacgctaagactaagccaagagaagagcagtacaactccacttacagagttgtttccgttttgactgttttgcaccaggattggttgaacggtaaagaatacaagtgtaaggtttccaacaaggctttgccagctccaatcgaaaagactatctccaaggctaagggtcaaccaagagagccacaggtttacactttgccaccatccagagatgagttgactaagaaccaggtttccttgacttgtttggttaagggattctacccatccgacattgctgttgagtgggaatctaacggtcaaccagagaacaactacaagactactccaccagttttggattctgacggttccttcttcttgtactccaagttgactgttgacaagtccagatggcaacagggtaacgttttctcctgttccgttatgcatgaggctttgaagtttcactacactcaaaagtccttgtctttgtcccctggtaagtaatga

SEQ ID NO: 14 - protein, 252 aa, Artificial Sequence
  Human IgG1 Fc region containing mutations
MRFPSIFTAVLFAASSALAAEPKSCDKTHTCPPCPAPELLGGPSVFLFPPKPKDTLYITREPEVTCVVAAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 15 - DNA, 762 bp, Artificial Sequence
  Human IgG1 Fc region containing mutations
atgagatttccttcaatttttactgctgttttattcgcagcatcctccgcattagctgctgaaccaaagtcttgtgacaagacacacacttgtccaccatgtccagctccagaattgttgggtggtccatccgtttttttgtttccaccaaagccaaaggacactttgtacatcactagagaaccagaggttacatgtgttgttgttgctgtttctcacgaggacccagaggttaagttcaactggtacgttgacggtgttgaagttcacaacgctaagactaagccaagagaagagcagtacaactccacttacgctgttgtttccgttttgactgttttgcaccaggattggttgaacggtaaagaatacaagtgtaaggtttccaacaaggctttgccagctccaatcgaaaagactatctccaaggctaagggtcaaccaagagagccacaggtttacactttgccaccatccagagatgagttgactaagaaccaggtttccttgacttgtttggttaagggattctacccatccgacattgctgttgagtgggaatctaacggtcaaccagagaacaactacaagactactccaccagttttggattctgacggttccttcttcttgtactccaagttgactgttgacaagtccagatggcaacagggtaacgttttctcctgttccgttatgcatgaggctttgaagtttcactacactcaaaagtccttgtctttgtcccctggtaagtaatga

SEQ ID NO: 16 - protein, 252 aa, Artificial Sequence
  Human IgG1 Fc region comprising mutations
MRFPSIFTAVLFAASSALAAEPKSCDKTHTCPPCPAPELLGGPSVFLFPPKPKDTLYITREPEVTCVVVAVSHEDPEVKFNWYVDGVEVHNAKTKPREEQYNSTYAVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 17 - protein, 451 aa, Artificial Sequence
  Heavy chain antibody sequence containing mutations in the Fc region
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLAPPKPKDTLYITREPEVTCVVADVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 18 - protein, 214 aa, Artificial Sequence
  Light chain antibody sequence containing mutations
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - DNA, 699 bp, Artificial Sequence
  Human IgG1 Fc region containing mutations
gctgaaccaaagtcttgtgacaagacacacacttgtccaccatgtccagctccagaattgttgggtggtccatccgtttttttggctccaccaaagccaaaggacactttgtacatcactagagaaccagaggttacatgtgttgttgctgacgtttctcacgaggacccagaggttaagttcaactggtacgttgacggtgttgaagttcacaacgctaagactaagccaagagaagagcagtacaactccacttacagagttgtttccgttttgactgttttgcaccaggattggttgaacggtaaagaatacaagtgtaaggtttccaacaaggctttgccagctccaatcgaaaagactatctccaaggctaagggtcaaccaagagagccacaggtttacactttgccaccatccagagatgagttgactaagaaccaggtttccttgacttgtttggttaagggattctacccatccgacattgctgttgagtgggaatctaacggtcaaccagagaacaactacaagactactccaccagttttggattctgacggttccttcttcttgtactccaagttgactgttgacaagtccagatggcaacagggtaacgttttctcctgttccgttatgcatgaggctttgaagtttcactacactcaaaagtccttgtctttgtcccctggtaag

SEQ ID NO: 20 - protein, 233 aa, Artificial Sequence
  Human IgG1 Fc region containing mutations
AEPKSCDKTHTCPPCPAPELLGGPSVFLAPPKPKDTLYITREPEVTCVVADVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 21 - DNA, 699 bp, Artificial Sequence
  Human IgG1 Fc region containing mutations
gctgaaccaaagtcttgtgacaagacacacacttgtccaccatgtccagctccagaattgttgggtggtccatccgtttttttgtttccaccaaagccaaaggacactttgtacatcactagagaaccagaggttacatgtgttgttgttgacgtttctcacgaggacccagaggttaagttcaactggtacgttgacggtgttgaagttcacaacgctaagactaagccaagagaagagcagtacaactccacttacagagttgtttccgttttgactgttttgcaccaggattggttgaacggtaaagaatacaagtgtaaggtttccaacaaggctttgccagctccaatcgaaaagactatctccaaggctaagggtcaaccaagagagccacaggtttacactttgccaccatccagagatgagttgactaagaaccaggtttccttgacttgtttggttaagggattctacccatccgacattgctgttgagtgggaatctaacggtcaaccagagaacaactacaagactactccaccagttttggattctgacggttccttcttcttgtactccaagttgactgttgacaagtccagatggcaacagggtaacgttttctcctgttccgttatgcatgaggctttgaagtttcactacactcaaaagtccttgtctttgtcccctggtaag

SEQ ID NO: 22 - protein, 233 aa, Artificial Sequence
  Human IgG1 region containing mutations
AEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 23 - DNA, 699 bp, Artificial Sequence
  Human IgG1 Fc region comprising mutations
gctgaaccaaagtcttgtgacaagacacacacttgtccaccatgtccagctccagaattgttgggtggtccatccgtttttttggctccaccaaagccaaaggacactttgatgatctccagaactccagaggttacatgtgttgttgctgacgtttctcacgaggacccagaggttaagttcaactggtacgttgacggtgttgaagttcacaacgctaagactaagccaagagaagagcagtacaactccacttacagagttgtttccgttttgactgttttgcaccaggattggttgaacggtaaagaatacaagtgtaaggtttccaacaaggctttgccagctccaatcgaaaagactatctccaaggctaagggtcaaccaagagagccacaggtttacactttgccaccatccagagatgagttgactaagaaccaggtttccttgacttgtttggttaagggattctacccatccgacattgctgttgagtgggaatctaacggtcaaccagagaacaactacaagactactccaccagttttggattctgacggttccttcttcttgtactccaagttgactgttgacaagtccagatggcaacagggtaacgttttctcctgttccgttatgcatgaggctttgcacaaccactacactcaaaagtccttgtctttgtcccctggtaag

SEQ ID NO: 24 - protein, 233 aa, Artificial Sequence
  Human IgG1 Fc region containing mutations
AEPKSCDKTHTCPPCPAPELLGGPSVFLAPPKPKDTLMISRTPEVTCVVADVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 451 aa, Artificial Sequence
  Heavy chain antibody sequence containing mutations in the Fc region
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLAPPKPKDTLMISRTPEVTCVVADVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 451 aa, Artificial Sequence
  Heavy chain antibody sequence containing mutations in Fc region
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 27 - protein, 222 aa, Artificial Sequence
  Human IgG1 Fc region
TCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 28 - protein, 232 aa, Artificial Sequence
  Human IgG1 Fc region
AEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

(19) United States
(12) Patent Application Publication
(10) Pub. No.: US 2014/0302028 A1
(43) Pub. Date: Oct. 9, 2014

(54) FC CONTAINING POLYPEPTIDES HAVING INCREASED ANTI-INFLAMMATORY PROPERTIES AND INCREASED FCRN BINDING

(71) Applicant: Merck Sharp & Dohme Corp., Rahway, NJ (US)

(72) Inventor: Dongxing Zha, Etna, NH (US)

(21) Appl. No.: 14/357,779

(22) PCT Filed: Nov. 14, 2012
(86) PCT No.: PCT/US2012/064972

§ 371 Date: May 13, 2014

Related U.S. Application Data
(60) Provisional application No. 61/561,412, filed on Nov. 18, 2011.

(51) Int. Cl. C07K 16/46; C07K 16/18

(52) U.S. Cl. CPC C07K 16/46; C07K 16/18
USPC 424/134.1; 530/387.3; 424/172.1; 530/387.1; 435/69.6

(57) ABSTRACT

The present invention is directed to methods and compositions for the production of Fc-containing polypeptides which are useful as human or animal therapeutic agents, and which comprise increased anti-inflammatory properties and improved FcRn binding.

### FIELD OF THE INVENTION

The present invention is directed to methods and compositions for the production of Fc-containing polypeptides which are useful as human or animal therapeutic agents, and which comprise increased anti-inflammatory properties and improved FcRn binding.

### BACKGROUND OF THE INVENTION

Monoclonal antibodies often achieve their therapeutic benefit through two binding events. First, the variable domain of the antibody binds a specific protein on a target cell, for example, CD20 on the surface of cancer cells. This is followed by recruitment of effector cells such as natural killer (NK) cells that bind to the constant region (Fc) of the antibody and destroy cells to which the antibody is bound. This process, known as antibody-dependent cell cytotoxicity (ADCC), depends on a specific N-glycosylation event at Asn 297 in the Fc domain of the heavy chain of IgG1s, Rothman et al., Mol. Immunol. 26: 1113-1123 (1989). Antibodies that lack this N-glycosylation structure still bind antigen but cannot mediate ADCC, apparently as a result of reduced affinity of the Fc domain of the antibody for the Fc Receptor FcγRIIIa on the surface of NK cells.

The presence of N-glycosylation not only plays a role in the effector function of an antibody, the particular composition of the N-linked oligosaccharide is also important for its end function. The lack of fucose or the presence of bisecting N-acetyl glucosamine has been positively correlated with the potency of the ADCC, Rothman (1989), Umana et al., Nat. Biotech. 17: 176-180 (1999), Shields et al., J. Biol. Chem. 277: 26733-26740 (2002), and Shinkawa et al., J. Biol. Chem. 278: 3466-3473 (2003). There is also evidence that sialylation in the Fc region is positively correlated with the anti-inflammatory properties of intravenous immunoglobulin (IVIG). See, e.g., Kaneko et al., Science, 313: 670-673, 2006; Nimmerjahn and Ravetch., J. Exp. Med., 204: 11-15, 2007.

Given the utility of specific N-glycosylation in the function and potency of antibodies, a method for modifying the composition of N-linked oligosaccharides and modifying the properties of antibodies would be desirable.

A class of antibodies known as “Abdegs” have been engineered to bind with increased affinity to the neonatal FcR (FcRn) receptor. Patel et al., J. Immunol., 187:1015-1022 (2011). It has been postulated that these antibodies can be used for the treatment of autoimmune diseases. Methods of improving the biological properties of these antibodies would also be desirable.

Yeast and other fungal hosts are important production platforms for the generation of recombinant proteins. Yeasts are eukaryotes and, therefore, share common evolutionary processes with higher eukaryotes, including many of the post-translational modifications that occur in the secretory pathway. Recent advances in glycoengineering have resulted in cell lines of the yeast strain Pichia pastoris with genetically modified glycosylation pathways that allow them to carry out a sequence of enzymatic reactions, which mimic the process of glycosylation in humans. See, for example, U.S. Pat. Nos. 7,029,872, 7,326,681 and 7,449,308 that describe methods for producing a recombinant glycoprotein in a lower eukaryote host cell that are substantially identical to their human counterparts. Human-like sialylated bi-antennary complex N-linked glycans like those produced in Pichia pastoris from the aforesaid methods have demonstrated utility for the production of therapeutic glycoproteins. Thus, a method for further modifying or improving the production of antibodies in yeasts such as Pichia pastoris would be desirable.

### SUMMARY OF THE INVENTION

### Fc-Containing Polypeptides

The invention relates to an Fc-containing polypeptide comprising mutations at amino acid positions 252, 254, 256, 433, 434, 243 and 264 of the Fc region, wherein the numbering is according to the EU index as in Kabat, and wherein the Fc-containing polypeptide comprises sialylated N-glycans. In one embodiment, the sialic acid residues in the sialylated N-glycans are attached via α-2,6 linkages. In one embodiment, the Fc-containing polypeptide further comprises mutations at positions 267 and 338.

In one embodiment, the mutations at position 252, 254, 256, 433 and 434 are: M252Y, S254T, T256E, H433K and N434F.

In one embodiment, the mutations at positions 243 are selected from the group consisting of: F243A, F243G, F243S, F243T, F243V, F243L, F243I, F243D, F243Y, F243E, F243R, F243W and F243K.

In one embodiment, the mutations at position 264 are selected from the group consisting of: V264A, V264G, V264S, V264T, V264D, V264E, V264K, V264W, V264H, V264P, V264N, V264Q and V264L.

In one embodiment, the mutations at positions 243 and 264 are selected from the group consisting of: a) F243A and V264A; b) F243Y and V264G; c) F243T and V264G; d) F243L and V264A; f) F243L and V264N; and g) F243V and V264G.

In one embodiment, the Fc-containing polypeptide comprises mutations: M252Y, S254T, T256E, H433K, N434F, F243A and V264A.

In one embodiment, the Fc-containing polypeptide comprises mutations: M252Y, S254T, T256E, H433K, N434F, F243A, V264A, S267E and L328F.

The invention also comprises an Fc-containing polypeptide comprising SEQ ID NO:2 (or a fragment thereof corresponding to the Fc region as defined in SEQ ID NO:27 or SEQ ID NO:28).

The invention also comprises an Fc-containing polypeptide comprising SEQ ID NO:4 (or a fragment thereof corresponding to the Fc region as defined in SEQ ID NO:27 or SEQ ID NO:28).

The invention also comprises an Fc-containing polypeptide comprising SEQ ID NO:6 (or a fragment thereof corresponding to the Fc region as defined in SEQ ID NO:27 or SEQ ID NO:28).

The invention also comprises an Fc-containing polypeptide comprising SEQ ID NO:8 (or a fragment thereof corresponding to the Fc region as defined in SEQ ID NO:27 or SEQ ID NO:28).

The invention also comprises an Fc-containing polypeptide comprising SEQ ID NO:10 (or a fragment thereof corresponding to the Fc region as defined in SEQ ID NO:27 or SEQ ID NO:28).

The invention also comprises an Fc-containing polypeptide comprising SEQ ID NO:12 (or a fragment thereof corresponding to the Fc region as defined in SEQ ID NO:27 or SEQ ID NO:28).

The invention also comprises an Fc-containing polypeptide comprising SEQ ID NO:14 (or a fragment thereof corresponding to the Fc region as defined in SEQ ID NO:27 or SEQ ID NO:28).

The invention also comprises an Fc-containing polypeptide comprising SEQ ID NO:16 (or a fragment thereof corresponding to the Fc region as defined in SEQ ID NO:27 or SEQ ID NO:28).

The invention also comprises an Fc-containing polypeptide comprising SEQ ID NO:17 (or a fragment thereof corresponding to the Fc region as defined in SEQ ID NO:27 or SEQ ID NO:28).

The invention also comprises an Fc-containing polypeptide comprising SEQ ID NO:20 (or a fragment thereof corresponding to the Fc region as defined in SEQ ID NO:27 or SEQ ID NO:28).

In one embodiment, at least 30%, 40%, 50%, 60%, 70%, 80% or 90% of the N-glycans on the Fc-containing polypeptide comprise an N-linked oligosaccharide structure selected from the group consisting of SA(1-4)Gal(1-4)GlcNAc(2-4)Man3GlcNAc2. In one embodiment, at least 30%, 40%, 50%, 60%, 70%, 80% or 90% of the N-glycans on the Fc-containing polypeptide comprise an N-linked oligosaccharide structure selected from the group consisting of SA2Gal2GlcNAc2Man3GlcNAc2. In one embodiment, at least 30%, 40%, 50%, 60%, 70%, 80% or 90% of the N-glycans on the Fc-containing polypeptide comprise an N-linked oligosaccharide structure selected from the group consisting of NANA2Gal2GlcNAc2Man3GlcNAc2.

In one embodiment, the Fc-containing polypeptide is an antibody or an antibody fragment, wherein at least 30%, 40%, 50%, 60%, 70%, 80% or 90% of the N-glycans on the antibody or antibody fragment comprise an N-linked oligosaccharide structure selected from the group consisting of SA(1-4)Gal(1-4)GlcNAc(2-4)Man3GlcNAc2. In one embodiment, at least 30%, 40%, 50%, 60%, 70%, 80% or 90% of the N-glycans on the antibody or antibody fragment comprise an N-linked oligosaccharide structure selected from the group consisting of SA2Gal2GlcNAc2Man3GlcNAc2. In one embodiment, at least 30%, 40%, 50%, 60%, 70%, 80% or 90% of the N-glycans on the Fc antibody or antibody fragment comprise an N-linked oligosaccharide structure selected from the group consisting of NANA2Gal2GlcNAc2Man3GlcNAc2. In one embodiment, the Fc-containing polypeptide is an IgG1 subtype or a fragment thereof. In one embodiment, the Fc-containing polypeptide is an IgG3 subtype or a fragment thereof. In one embodiment, the Fc-containing polypeptide is an IgG2 subtype or a fragment thereof. In one embodiment, the Fc-containing polypeptide is an IgG4 subtype or a fragment thereof.

In one embodiment, the Fc-containing polypeptide has increased FcRn binding and has one or more of the following properties when compared to a parent Fc-containing polypeptide: a) reduced effector function, b) increased anti-inflammatory properties, c) increased sialylation, d) increased bioavailability when administered parenterally, e) reduced binding to FcγRI, FcγRIIa and FcγRIIIa, f) increased binding to FcγRIIb; and g) increased affinity to human FcRn at pH6 and pH7. In one embodiment, the parent polypeptide refers to an Fc-containing polypeptide which lacks mutations in the Fc region. In one embodiment, the parent polypeptide refers to an Fc-containing polypeptide which lacks mutations at positions 252, 254, 256, 433, 434, 243 and 264, wherein the number is according to the EU index as in Kabat. In another embodiment, the parent polypeptide refers to an Fc-containing polypeptide which comprises mutations at positions 252, 254, 256, 433, 434 but lacks mutations at positions 243 and 264.

As discussed above, the Fc-containing polypeptide of the invention comprises sialylated N-glycans (having a structure selected from SA₍₁₋₄₎Gal₍₁₋₄₎GlcNAc₍₂₋₄₎Man₃GlcNAc₂ or SAGalGlcNAcMan5GlNAc₂). The sialic acid residues may include NANA, NGNA, and analogs and derivatives thereof. In one embodiment, the Fc-containing polypeptides of the invention comprise a mixture of α-2,3 and α-2,6 linked sialic acid. In another embodiment, the Fc-containing polypeptides of the invention comprise only α-2,6 linked sialic acid. In one embodiment, the Fc-containing polypeptides of the invention comprise α-2,6 linked sialic acid and comprise no detectable level of α-2,3 linked sialic acid. In one embodiment, the sialic acid is N-acetylneuraminic acid (NANA) or N-glycolylneuraminic acid (NGNA) or a mixture thereof. In another embodiment, the sialic acid is an analog or derivative of NANA or NGNA with acetylation at position 9 on the sialic acid. In one embodiment, the N-glycans on the Fc-containing polypeptides of the invention comprise NANA and no NGNA.

In one embodiment, the Fc-containing polypeptide comprises N-glycans comprising sialic acid (including NANA, NGNA, and analogs and derivatives thereof). In one embodiment, the Fc-containing polypeptide produced by the claimed method has an N-glycan composition in which at least 40 mole %, 70 mole % or 90 mole % of the N-glycans on the Fc-containing polypeptide are sialylated (have a structure selected from SA₍₁₋₄₎Gal₍₁₋₄₎GlcNAc₍₂₋₄₎Man₃GlcNAc₂ or SAGalGlcNAcMan5GlNAcc₂). In one embodiment, least 47 mole % of the N-glycans on the Fc-containing polypeptides have the structure SA₂Gal₂GlcNAc₂Man₃GlcNAc₂. In another embodiment, least 47 mole % of the N-glycans on the Fc-containing polypeptides have the structure NANA₂Gal₂GlcNAc₂Man₃GlcNAc₂. In another embodiment, least 66 mole % of the N-glycans on the Fc-containing polypeptides have the structure SA₂Gal₂GlcNAc₂Man₃GlcNAc2. In another embodiment, least 66 mole % of the N-glycans on the Fc-containing polypeptides have the structure NANA₂Gal₂GlcNAc₂Man₃GlcNAc2.

The N-glycans on the Fc-containing polypeptides of the invention can optionally comprise fucose. In one embodiment, the N-glycans on the Fc-containing polypeptides will comprise a mixture of fucosylated and non-fucosylated N-glycans. In another embodiment, the N-glycans on the Fc-containing polypeptides lack fucose.

The invention also comprises a pharmaceutical composition comprising any of the above described Fc-containing polypeptides and a pharmaceutically acceptable carrier.

### Methods of Producing Fc-Containing Polypeptides

The invention also comprises a method for producing a Fc-containing polypeptide in a host cell comprising: a) providing a genetically modified host cell that has been engineered to produce an Fc-containing polypeptide comprising sialylated N-glycans, wherein the host cell comprises a nucleic acid encoding mutations at amino acid positions 252, 254, 256, 433, 434, 243 and 264 of the Fc region, wherein the numbering is according to the EU index as in Kabat; b) culturing the host cell under conditions which cause expression of the Fc-containing polypeptide; and c) isolating the Fc-containing polypeptide from the host cell. In one embodiment, the nucleic acid further encodes mutations at amino acid positions 267 and 338.

In one embodiment, the mutations at position 252, 254, 256, 433 and 434 are: M252Y, S254T, T256E, H433K and N434F.

In one embodiment, the mutations at positions 243 are selected from the group consisting of: F243A, F243G, F243S, F243T, F243V, F243L, F243I, F243D, F243Y, F243E, F243R, F243W and F243K.

In one embodiment, the mutations at position 264 are selected from the group consisting of: V264A, V264G, V264S, V264T, V264D, V264E, V264K, V264W, V264H, V264P, V264N, V264Q and V264L.

In one embodiment, the mutations at positions 243 and 264 are selected from the group consisting of: a) F243A and V264A; b) F243Y and V264G; c) F243T and V264G; d) F243L and V264A; f) F243L and V264N; and g) F243V and V264G.

In one embodiment, the nucleic acid encodes mutations: M252Y, S254T, T256E, H433K, N434F, F243A and V264A.

In one embodiment, the Fc nucleic acid encodes mutations: M252Y, S254T, T256E, H433K, N434F, F243A, V264A, S267E and L328F.

The invention also comprises a method for producing a Fc-containing polypeptide in a host cell comprising: a) providing a genetically modified host cell capable of producing a polypeptide comprising sialylated N-glycans, wherein the cell has been engineered to produce an Fc-containing polypeptide comprising any one of the Fc mutation combinations identified in Table 1 of Example 1; b) culturing the host cell under conditions which cause expression of the Fc-containing polypeptide; and c) isolating the Fc-containing polypeptide from the host cell. In one embodiment, the nucleic acid further encodes mutations at amino acid positions 267 and 338.

In one embodiment, at least 30%, 40%, 50%, 60%, 70%, 80% or 90% of the N-glycans on the Fc-containing polypeptide comprise an N-linked oligosaccharide structure selected from the group consisting of SA(1-4)Gal(1-4)GlcNAc(2-4)Man3GlcNAc2. In one embodiment, at least 30%, 40%, 50%, 60%, 70%, 80% or 90% of the N-glycans on the Fc-containing polypeptide comprise an N-linked oligosaccharide structure selected from the group consisting of SA2Gal2GlcNAc2Man3GlcNAc2. In one embodiment, at least 30%, 40%, 50%, 60%, 70%, 80% or 90% of the N-glycans on the Fc-containing polypeptide comprise an N-linked oligosaccharide structure selected from the group consisting of NANA2Gal2GlcNAc2Man3GlcNAc2.

In one embodiment, the Fc-containing polypeptide is an antibody or an antibody fragment, wherein at least 30%, 40%, 50%, 60%, 70%, 80% or 90% of the N-glycans on the antibody or antibody fragment comprise an N-linked oligosaccharide structure selected from the group consisting of SA(1-4)Gal(1-4)GlcNAc(2-4)Man3GlcNAc2. In one embodiment, at least 30%, 40%, 50%, 60%, 70%, 80% or 90% of the N-glycans on the antibody or antibody fragment comprise an N-linked oligosaccharide structure selected from the group consisting of SA2Gal2GlcNAc2Man3GlcNAc2. In one embodiment, at least 30%, 40%, 50%, 60%, 70%, 80% or 90% of the N-glycans on the Fc antibody or antibody fragment comprise an N-linked oligosaccharide structure selected from the group consisting of NANA2Gal2GlcNAc2Man3GlcNAc2. In one embodiment, the Fc-containing polypeptide is of an IgG1 subtype or a fragment thereof. In one embodiment, the Fc-containing polypeptide is an IgG3 subtype or a fragment thereof. In one embodiment, the Fc-containing polypeptide is an IgG2 subtype or a fragment thereof. In one embodiment, the Fc-containing polypeptide is an IgG4 subtype or a fragment thereof.

In one embodiment, the Fc-containing polypeptide of the invention has an N-glycan composition in which the amount and percentage of total sialylated N-glycans is increased relative to a parent Fc-containing polypeptide. In one embodiment, the parent polypeptide refers to an Fc-containing polypeptide which lacks mutations in the Fc region. In one embodiment, the parent polypeptide refers to an Fc-containing polypeptide which lacks mutations at positions 252, 254, 256, 433, 434, 243 and 264, wherein the number is according to the EU index as in Kabat. In another embodiment, the parent polypeptide refers to an Fc-containing polypeptide which comprises mutations at positions 252, 254, 256, 433, 434 but lacks mutations at positions 243 and 264.

In one embodiment, the Fc-containing polypeptide of the invention has increased FcRn binding and has one or more of the following properties when compared to a parent Fc-containing polypeptide: a) reduced effector function, b) increased anti-inflammatory properties, c) increased sialylation, d) increased bioavailability when administered parenterally, e) reduced binding to FcγRI, FcγRIIa and FcγRIIIa, f) increased binding to FcγRIIb; and g) increased affinity to human FcRn at pH6 and pH7. In one embodiment, the parent polypeptide refers to an Fc-containing polypeptide which lacks mutations in the Fc region. In one embodiment, the parent polypeptide refers to an Fc-containing polypeptide which lacks mutations at positions 252, 254, 256, 433, 434, 243 and 264, wherein the number is according to the EU index as in Kabat. In another embodiment, the parent polypeptide refers to an Fc-containing polypeptide which comprises mutations at positions 252, 254, 256, 433, 434 but lacks mutations at positions 243 and 264.

As discussed above, the Fc-containing polypeptide of the invention comprises sialylated N-glycans (including NANA, NGNA, and analogs and derivatives thereof). In one embodiment, the Fc-containing polypeptides of the invention comprise a mixture of α-2,3 and α-2,6 linked sialic acid. In another embodiment, the Fc-containing polypeptides of the invention comprise only α-2,6 linked sialic acid. In one embodiment, the Fc-containing polypeptides of the invention comprise α-2,6 linked sialic acid and comprise no detectable level of α-2,3 linked sialic acid. In one embodiment, the sialic acid is N-acetylneuraminic acid (NANA) or N-glycolylneuraminic acid (NGNA) or a mixture thereof. In another embodiment, the sialic acid is an analog or derivative of NANA or NGNA with acetylation at position 9 on the sialic acid. In one embodiment, the N-glycans on the Fc-containing polypeptides of the invention comprise NANA and no NGNA.

The N-glycans on the Fc-containing polypeptides of the invention can optionally comprise fucose. In one embodiment, the N-glycans on the Fc-containing polypeptides will comprise a mixture of fucosylated and non-fucosylated N-glycans. In another embodiment, the N-glycans on the Fc-containing polypeptides lack fucose.

In one embodiment, the method for producing an Fc-containing polypeptide is carried out in a mammalian cell. In another embodiment, the method for producing an Fc-containing polypeptide is carried out in a plant cell. In another embodiment, the method for producing an Fc-containing polypeptide is carried out in bacteria. In another embodiment, the method for producing an Fc-containing polypeptide is carried out in an insect cell. In another embodiment, the method for producing an Fc-containing polypeptide is carried out in a lower eukaryotic cell. In another embodiment, the method for producing an Fc-containing polypeptide is carried out in a yeast cell. In one embodiment, the method for producing an Fc-containing polypeptide is carried out in Pichia pastoris.

In one embodiment, the Fc-containing polypeptide produced by the claimed method comprises N-glycans comprising sialic acid (including NANA, NGNA, and analogs and derivatives thereof). In one embodiment, the Fc-containing polypeptide produced by the claimed method has an N-glycan composition in which at least 40 mole %, 70 mole % or 90 mole % of the N-glycans on the Fc-containing polypeptide are sialylated (have a structure selected from SA₍₁₋₄₎Gal₍₁₋₄₎GlcNAc₍₂₋₄₎Man₃GlcNAc₂ or SAGalGlcNAcMan5GlNAcc₂). In one embodiment, least 47 mole % of the N-glycans on the Fc-containing polypeptides have the structure SA₂Gal₂GlcNAc₂Man₃GlcNAc2. In another embodiment, least 47 mole % of the N-glycans on the Fc-containing polypeptides have the structure NANA₂Gal₂GlcNAc₂Man₃GlcNAc2. In another embodiment, least 66 mole % of the N-glycans on the Fc-containing polypeptides have the structure SA₂Gal₂GlcNAc₂Man₃GlcNAc₂. In another embodiment, least 66 mole % of the N-glycans on the Fc-containing polypeptides have the structure NANA₂Gal₂GlcNAc₂Man₃GlcNAc2. In one embodiment, the Fc-containing polypeptides produced by the claimed method comprise a mixture of α-2,3 and α-2,6 linked sialic acid. In another embodiment, the Fc-containing polypeptides comprise only α-2,6 linked sialic acid. In one embodiment, the Fc-containing polypeptides of the invention comprise α-2,6 linked sialic acid and comprise no detectable level of α-2,3 linked sialic acid. In one embodiment, the sialic acid is N-acetylneuraminic acid (NANA) or N-glycolylneuraminic acid (NGNA) or a mixture thereof. In another embodiment, the sialic acid is an analog or derivative of NANA or NGNA with acetylation at position 9 on the sialic acid. In one embodiment, the N-glycans on the Fc-containing polypeptides produced by the claimed method comprise NANA and no NGNA.

In one embodiment, the Fc-containing polypeptide produced by the claimed method has an N-glycan composition in which the amount and percentage of total sialylated N-glycans is increased relative to a parent Fc-containing polypeptide. In one embodiment, the parent polypeptide refers to an Fc-containing polypeptide which lacks mutations in the Fc region. In one embodiment, the parent polypeptide refers to an Fc-containing polypeptide which lacks mutations at positions 252, 254, 256, 433, 434, 243 and 264, wherein the number is according to the EU index as in Kabat. In another embodiment, the parent polypeptide refers to an Fc-containing polypeptide which comprises mutations at positions 252, 254, 256, 433, 434 but lacks mutations at positions 243 and 264.

### Biological Uses of Fc-Containing Polypeptides

The invention also comprises a method of increasing the anti-inflammatory properties or decreasing cytotoxicity of an Fc-containing polypeptide comprising introducing mutations at positions 252, 254, 256, 433, 434, 243 and 264 of the Fc region, wherein the numbering is according to the EU index as in Kabat; wherein the Fc-containing polypeptide has improved FcRn binding and increased anti-inflammatory properties or decreased cytotoxicity when compared to a parent Fc-containing polypeptide. In one embodiment, the Fc-containing polypeptide further comprises mutations at positions 267 and 338. In one embodiment, the mutations at position 252, 254, 256, 433 and 434 are: M252Y, S254T, T256E, H433K and N434F. In one embodiment, the mutations at positions 243 are selected from the group consisting of: F243A, F243G, F243S, F243T, F243V, F243L, F243I, F243D, F243Y, F243E, F243R, F243W and F243K. In one embodiment, the mutations at position 264 are selected from the group consisting of: V264A, V264G, V264S, V264T, V264D, V264E, V264K, V264W, V264H, V264P, V264N, V264Q and V264L. In one embodiment, the mutations at positions 243 and 264 are selected from the group consisting of: a) F243A and V264A; b) F243Y and V264G; c) F243T and V264G; d) F243L and V264A; f) F243L and V264N; and g) F243V and V264G. In one embodiment, the mutations are: M252Y, S254T, T256E, H433K, N434F, F243A and V264A. In one embodiment, the mutations are: M252Y, S254T, T256E, H433K, N434F, F243A, V264A, S267E and L328F. In one embodiment, the Fc-containing polypeptide is an antibody or an antibody fragment. In one embodiment, the Fc-containing polypeptide is an IgG1 subtype or a fragment thereof. In one embodiment, the Fc-containing polypeptide is an IgG3 subtype or a fragment thereof. In one embodiment, the Fc-containing polypeptide is an IgG2 subtype or a fragment thereof. In one embodiment, the Fc-containing polypeptide is an IgG4 subtype or a fragment thereof. In one embodiment, the parent polypeptide refers to an Fc-containing polypeptide which lacks mutations in the Fc region. In one embodiment, the parent polypeptide refers to an Fc-containing polypeptide which lacks mutations at positions 252, 254, 256, 433, 434, 243 and 264. In another embodiment, the parent polypeptide refers to an Fc-containing polypeptide which comprises mutations at positions 252, 254, 256, 433, 434 but lacks mutations at positions 243 and 264.

The invention also comprises a method of increasing the anti-inflammatory properties or decreasing cytotoxicity of an Fc-containing polypeptide comprising introducing any one of the mutation combinations identified in Table 1 of Example 1; wherein the Fc-containing polypeptide has improved FcRn binding and increased anti-inflammatory properties or decreased cytotoxicity when compared to a parent Fc-containing polypeptide. In one embodiment, the parent polypeptide refers to an Fc-containing polypeptide which lacks mutations in the Fc region. In one embodiment, the parent polypeptide refers to an Fc-containing polypeptide which lacks mutations at positions 252, 254, 256, 433, 434, 243 and 264, wherein the number is according to the EU index as in Kabat. In another embodiment, the parent polypeptide refers to an Fc-containing polypeptide which comprises mutations at positions 252, 254, 256, 433, 434 but lacks mutations at positions 243 and 264.

The invention also comprises a method of treating an inflammatory condition in a subject in need thereof comprising: administering to the subject a therapeutically effective amount of an Fc-containing polypeptide comprising mutations at positions 252, 254, 256, 433, 434, 243 and 264 of the Fc region, wherein the numbering is according to the EU index as in Kabat. In one embodiment, the Fc-containing polypeptide further comprises mutations at positions 267 and 338. In one embodiment, the mutations at position 252, 254, 256, 433 and 434 are: M252Y, S254T, T256E, H433K and N434F. In one embodiment, the mutations at positions 243 are selected from the group consisting of: F243A, F243G, F243S, F243T, F243V, F243L, F243I, F243D, F243Y, F243E, F243R, F243W and F243K. In one embodiment, the mutations at position 264 are selected from the group consisting of: V264A, V264G, V264S, V264T, V264D, V264E, V264K, V264W, V264H, V264P, V264N, V264Q and V264L, wherein the numbering is according to the EU index as in Kabat. In one embodiment, the mutations at positions 243 and 264 are selected from the group consisting of: a) F243A and V264A; b) F243Y and V264G; c) F243T and V264G; d) F243L and V264A; f) F243L and V264N; and g) F243V and V264G. In one embodiment, the mutations are: M252Y, S254T, T256E, H433K, N434F, F243A and V264A. In one embodiment, the mutations are: M252Y, S254T, T256E, H433K, N434F, F243A, V264A, S267E and L328F. In one embodiment, the Fc-containing polypeptide is an antibody or an antibody fragment. In one embodiment, the Fc-containing polypeptide is an IgG1 subtype or a fragment thereof. In one embodiment, the Fc-containing polypeptide is an IgG3 subtype or a fragment thereof. In one embodiment, the Fc-containing polypeptide is an IgG2 subtype or a fragment thereof. In one embodiment, the Fc-containing polypeptide is an IgG4 subtype or a fragment thereof.

The invention also comprises a method of treating an inflammatory condition in a subject in need thereof comprising: administering to the subject a therapeutically effective amount of an Fc-containing polypeptide comprising any one of the mutation combinations identified in Table 1 of Example 1.

In any of the above embodiments, an increase in anti-inflammatory activity can be detected using any method known in the art. In one embodiment, an increase in anti-inflammatory activity is detected by measuring a decrease in the expression of a gene selected from the group consisting of: IL-1β, IL-6, RANKL, TRAP, ATP6v0d2, MDL-1, DAP12, CD11b, TIMP-1, MMP9, CTSK, PU-1, MCP, MIP1α, Cxc11-Groa, CXcl2-Grob, CD18, TNF, FcγRI, FcγRIb, FcγRIII and FcγRIV.

In any embodiments, the Fc-containing polypeptide is an antibody that targets human FcRn.

### BRIEF DESCRIPTION OF THE DRAWINGS

FIG. 1 illustrates the plasmid designated pGLY4464.

FIG. 2 illustrates the plasmid designated pGLY11544.

### DETAILED DESCRIPTION OF THE INVENTION

### Definitions

The term “G0” when used herein refers to a complex bi-antennary oligosaccharide without galactose or fucose, GlcNAc₂Man₃GlcNAc₂.

The term “G1” when used herein refers to a complex bi-antennary oligosaccharide without fucose and containing one galactosyl residue, GalGlcNAc₂Man₃GlcNAc₂.

The term “G2” when used herein refers to a complex bi-antennary oligosaccharide without fucose and containing two galactosyl residues, Gal₂GlcNAc₂Man₃GlcNAc₂.

The term “G0F” when used herein refers to a complex bi-antennary oligosaccharide containing a core fucose and without galactose, GlcNAc₂Man₃GlcNAc₂F.

The term “G1F” when used herein refers to a complex bi-antennary oligosaccharide containing a core fucose and one galactosyl residue, GalGlcNAc₂Man₃GlcNAc₂F.

The term “G2F” when used herein refers to a complex bi-antennary oligosaccharide containing a core fucose and two galactosyl residues, Gal₂GlcNAc₂Man₃GlcNAc₂F.

The term “Man5” when used herein refers to the oligosaccharide structure shown as

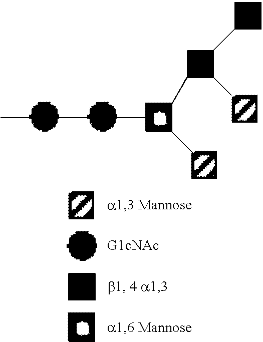

The term “GFI 5.0” when used herein refers to glycoengineered Pichia pastoris strains that produce glycoproteins having predominantly Gal₂GlcNAc₂Man₃GlcNAc₂ N-glycans.

The term “GFI 6.0” when used herein refers to glycoengineered Pichia pastoris strains that produce glycoproteins having predominantly NANA₂Gal₂GlcNAc₂Man₃GlcNAc₂ N-glycans.

The term “GS5.0”, when used herein refers to the N-glycosylation structure Gal₂GlcNAc₂Man₃GlcNAc₂.

The term “GS5.5”, when used herein refers to the N-glycosylation structure NANAGal₂GlcNAc₂Man₃GlcNAc₂, which when produced in Pichia pastoris strains to which α 2,6 sialyl transferase has been glycoengineered result in α-2,6-linked sialic acid and which when produced in Pichia pastoris strains to which α-2,3 sialyl transferase has been glycoengineered result in α-2,3-linked sialic acid.

The term “GS6.0”, when used herein refers to the N-glycosylation structure NANA₂Gal₂GlcNAc₂Man₃GlcNAc₂, which when produced in Pichia pastoris strains to which α-2,6 sialyltransferase has been glycoengineered result in α-2,6-linked sialic acid and which when produced in Pichia pastoris strains to which α-2,3 sialyl transferase has been glycoengineered result in α-2,3-linked sialic acid.

The term “wild type” or “wt” when used herein in connection to a Pichia pastoris strain refers to a native Pichia pastoris strain that has not been subjected to genetic modification to control glycosylation.

The term “antibody”, when used herein refers to an immunoglobulin molecule capable of binding to a specific antigen through at least one antigen recognition site located in the variable region of the immunoglobulin molecule. As used herein, the term encompasses not only intact polyclonal or monoclonal antibodies, consisting of four polypeptide chains, i.e. two identical pairs of polypeptide chains, each pair having one “light” chain (LC) (about 25 kDa) and one “heavy” chain (HC) (about 50-70 kDa), but also fragments thereof, such as Fab, Fab′, F(ab′)₂, Fv, single chain (ScFv), mutants thereof, fusion proteins comprising an antibody portion, and any other modified configuration of an immunoglobulin molecule that comprises an antigen recognition site and at least the portion of the C_{H}2 domain of the heavy chain immunoglobulin constant region which comprises an N-linked glycosylation site of the C_{H}2 domain, or a variant thereof. As used herein the term includes an antibody of any class, such as IgG (for example, IgG1, IgG2, IgG3 or IgG4), IgM, IgA, IgD and IgE, respectively.

The term “consensus sequence of C_{H}2” when used herein refers to the amino acid sequence of the C_{H}2 domain of the heavy chain constant region containing an N-linked glycosylation site which was derived from the most common amino acid sequences found in C_{H}2 domains from a variety of antibodies.

The term “Fc region” is used to define a C-terminal region of an immunoglobulin heavy chain. The “Fc region” may be a native sequence Fc region or a variant Fc region. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The Fc region of an immunoglobulin comprises two constant domains, CH2 and CH3, and can optionally comprise a hinge region. In one embodiment, the Fc region comprises the amino acid sequence of SEQ ID NO:27 (or a variant thereof comprising point mutations). In one embodiment, the Fc region comprises the amino acid sequence of SEQ ID NO:28 (or a variant thereof comprising point mutations). In another embodiment, the Fc region comprises the amino acid sequence of SEQ ID NO:27 or SEQ ID NO:28, with the addition of a lysine (K) residue at the 3′ end. The Fc region contains a single N-linked glycosylation site in the CH2 domain that corresponds to the Asn297 site of a full-length heavy chain of an antibody, wherein the numbering is according to the EU index as in Kaat.

The term “Fc-containing polypeptide” refers to a polypeptide, such as an antibody or immunoadhesin, which comprises an Fc region or fragment of an Fc region which retains the N-linked glycosylation site in the CH2 domain and retains the ability to recruit immune cells. This term encompasses polypeptides comprising or consisting of (or consisting essentially of) an Fc region either as a monomore or dimeric species. Polypeptides comprising an Fc region can be generated by papain digestion of antibodies or by recombinant DNA technology.

The term “parent antibody”, “parent immunoglobulin” or “parent Fc-containing polypeptide” when used herein refers to an antibody or Fc-containing polypeptide which lacks the Fc region mutations disclosed herein. A parent Fc-containing polypeptide may comprise a native sequence Fc region or an Fc region with pre-existing amino acid sequence modifications. A native sequence Fc region comprises an amino acid sequence identical to the amino acid sequence of an Fc region found in nature. Native sequence Fc regions include the native sequence human IgG1 Fc region, the native sequence human IgG2 Fc region, the native sequence human IgG3 Fc region and the native sequence human IgG4 Fc region as well as naturally occurring variants thereof. When used as a comparator, a parent antibody or a parent Fc-containing polypeptide can be expressed in any cell. In one embodiment, the parent antibody or a parent Fc-containing polypeptide is expressed in the same cell as the Fc-containing polypeptide of the invention.

As used herein, the term “immunoadhesin” designates antibody-like molecules which combine the “binding domain” of a heterologous “adhesin” protein (e.g. a receptor, ligand or enzyme) with an immunoglobulin constant domain. Structurally, the immunoadhesins comprise a fusion of the adhesin amino acid sequence with the desired binding specificity which is other than the antigen recognition and binding site (antigen combining site) of an antibody (i.e. is “heterologous”) and an immunoglobulin constant domain sequence. The term “ligand binding domain” as used herein refers to any native cell-surface receptor or any region or derivative thereof retaining at least a qualitative ligand binding ability of a corresponding native receptor. In a specific embodiment, the receptor is from a cell-surface polypeptide having an extracellular domain that is homologous to a member of the immunoglobulin supergenefamily. Other receptors, which are not members of the immunoglobulin supergenefamily but are nonetheless specifically covered by this definition, are receptors for cytokines, and in particular receptors with tyrosine kinase activity (receptor tyrosine kinases), members of the hematopoietin and nerve growth factor which predispose the mammal to the disorder in question. In one embodiment, the disorder is cancer. Methods of making immunoadhesins are well known in the art. See, e.g., WO00/42072.

The term “Fc mutein” or “Fc mutein antibody” when used herein refers to an Fc-containing polypeptide in which one or more point mutations have been made to the Fc region.

The term “Fc mutation” when used herein refers to a mutation made to the Fc region of an Fc-containing polypeptide.

Throughout the present specification and claims, the numbering of the residues in an immunoglobulin heavy chain or an Fc-containing polypeptide is that of the EU index as in Kabat et al., Sequences of Proteins of Immunological Interest, 5th Ed. Public Health Service, National Institutes of Health, Bethesda, Md. (1991), expressly incorporated herein by reference. The “EU index as in Kabat” refers to the residue numbering of the human IgG1 EU antibody.

The term “effector function” as used herein refers to a biochemical event that results from the interaction of an antibody Fc region with an Fc receptor or ligand. Exemplary “effector functions” include C1q binding; complement dependent cytotoxicity (CDC); Fc receptor binding; antibody-dependent cell-mediated cytotoxicity (ADCC); phagocytosis; down regulation of cell surface receptors (e. g. B cell receptor; BCR), etc. Such effector functions can be assessed using various assays known in the art.

The term “glycoengineered Pichia pastoris” when used herein refers to a strain of Pichia pastoris that has been genetically altered to express human-like N-glycans. For example, the GFI 5.0, GFI 5.5 and GFI 6.0 strains described above.

The terms “N-glycan”, “glycoprotein” and “glycoform” when used herein refer to an N-linked oligosaccharide, e.g., one that is attached by an asparagine-N-acetylglucosamine linkage to an asparagine residue of a polypeptide. Predominant sugars found on glycoproteins are glucose, galactose, mannose, fucose, N-acetylgalactosamine (GalNAc), N-acetylglucosamine (GlcNAc) and sialic acid (SA, including NANA, NGNA and derivatives and analogs thereof, including acetylated NANA or acetylated NGNA). In glycoengineered Pichia pastoris, sialic acid is exclusively N-acetyl-neuraminic acid (NANA) (Hamilton et al., Science 313 (5792): 1441-1443 (2006)). N-glycans have a common pentasaccharide core of Man₃GlcNAc₂, wherein “Man” refers to mannose, “Glc” refers to glucose, “NAc” refers to N-acetyl, and GlcNAc refers to N-acetylglucosamine. N-glycans differ with respect to the number of branches (antennae) comprising peripheral sugars (e.g., GlcNAc, galactose, fucose and sialic acid) that are added to the Man₃GlcNAc₂ (“Man3”) core structure which is also referred to as the “trimannose core”, the “pentasaccharide core” or the “paucimannose core”. N-glycans are classified according to their branched constituents (e.g., high mannose, complex or hybrid).

As used herein, the term “sialic acid” or “SA” refers to any member of the sialic acid family, including without limitation: N-acetylneuraminic acid (Neu5Ac or NANA), N-glycolylneuraminic acid (NGNA) and any analog or derivative thereof (including those arising from acetylation at any position on the sialic acid molecule). Sialic acid is a generic name for a group of about 30 naturally occurring acidic carbohydrates that are essential components of a large number of glycoconjugates. Schauer, Biochem. Society Transactions, 11, 270-271 (1983). Sialic acids are usually the terminal residue of the oligosaccharides. N-acetylneuraminic acid (NANA) is the most common sialic acid form and N-glycolylneuraminic acid (NGNA) is the second most common form. Schauer, Glycobiology, 1, 449-452 (1991). NGNA is widespread throughout the animal kingdom and, according to species and tissue, often constitutes a significant proportion of the glycoconjugate-bound sialic acid. Certain species such as chicken and man are exceptional, since they lack NGNA in normal tissues. Corfield, et al., Cell Biology Monographs, 10, 5-50 (1982). In human serum samples, the percentage of sialic acid in the form of NGNA is reported to be 0.01% of the total sialic acid. Schauer, “Sialic Acids as Antigenic Determinants of Complex Carbohydrates”, found in The Molecular Immunology of Complex Carbohydrates, (Plenum Press, New York, 1988).

The term “human-like N-glycan”, as used herein, refers to the N-linked oligosaccharides which closely resemble the oligosaccharides produced by non-engineered, wild-type human cells. For example, wild-type Pichia pastoris and other lower eukaryotic cells typically produce hypermannosylated proteins at N-glycosylation sites. The host cells described herein produce glycoproteins (for example, antibodies) comprising human-like N-glycans that are not hypermannosylated. In some embodiments, the host cells of the present invention are capable of producing human-like N-glycans with hybrid and/or complex N-glycans. The specific type of “human-like” glycans present on a specific glycoprotein produced from a host cell of the invention will depend upon the specific glycoengineering steps that are performed in the host cell.

The term “high mannose” type N-glycan when used herein refers to an N-glycan having five or more mannose residues.

The term “complex” type N-glycan when used herein refers to an N-glycan having at least one GlcNAc attached to the 1,3 mannose arm and at least one GlcNAc attached to the 1,6 mannose arm of a “trimannose” core. Complex N-glycans may also have galactose (“Gal”) or N-acetylgalactosamine (“GalNAc”) residues that are optionally modified with sialic acid or derivatives (e.g., “NANA” or “NeuAc”, where “Neu” refers to neuraminic acid and “Ac” refers to acetyl). Complex N-glycans may also have intrachain substitutions comprising “bisecting” GlcNAc and core fucose (“Fuc”). As an example, when a N-glycan comprises a bisecting GlcNAc on the trimannose core, the structure can be represented as Man₃GlcNAc₂(GlcNAc) or Man₃GlcNAc₃. When an N-glycan comprises a core fucose attached to the trimannose core, the structure may be represented as Man₃GlcNAc₂(Fuc). Complex N-glycans may also have multiple antennae on the “trimannose core,” often referred to as “multiple antennary glycans.”

The term “hybrid” N-glycan when used herein refers to an N-glycan having at least one GlcNAc on the terminal of the 1,3 mannose arm of the trimannose core and zero or more than one mannose on the 1,6 mannose arm of the trimannose core.

When referring to “mole percent” of a glycan present in a preparation of a glycoprotein, the term means the molar percent of a particular glycan present in the pool of N-linked oligosaccharides released when the protein preparation is treated with PNGase and then quantified by a method that is not affected by glycoform composition, (for instance, labeling a PNGase released glycan pool with a fluorescent tag such as 2-aminobenzamide and then separating by high performance liquid chromatography or capillary electrophoresis and then quantifying glycans by fluorescence intensity). For example, 50 mole percent NANA₂ Gal₂GlcNAc₂Man₃GlcNAc₂ means that 50 percent of the released glycans are NANA₂ Gal₂GlcNAc₂Man₃GlcNAc₂ and the remaining 50 percent are comprised of other N-linked oligosaccharides. Thus, in this application, the terms “mole percent” and “percent” are used interchangeably.

The term “anti-inflammatory antibody” as used herein, refers to an antibody intended to be used to treat inflammation. The anti-inflammatory properties of an Fc-containing polypeptide can be measured using any method known in the art. In one embodiment, the anti-inflammatory properties of an Fc-containing polypeptide are measured using an animal model, such as the models described in Kaneko et al., Science 313:670-673 (2006), Anthony et al., Science 320:373-376 (2008), and Examples 20-21 herein. In another embodiment, the anti-inflammatory properties of an Fc-containing polypeptide are measured by determining the level of a biomarker related to inflammation (including without limitation: CRP, pro-inflammatory cytokines such as tumor necrosis factors (TNF-alpha), interferon-gamma, interleukin 6 (IL-6, IL-8, IL-10, chemokines, the coagulation marker D-dimer, sCD14, intestinal fatty acid binding peptide (IFABP), and hyaluronic acid. In one embodiment, the anti-inflammatory properties of an Fc-containing polypeptide is measured by determining the level of C-reactive protein (CRP) using a method known in the art. A decrease in the level of C-reactive protein indicates that the Fc-containing polypeptide has anti-inflammatory properties.

“Conservatively modified variants” or “conservative substitution” refers to substitutions of amino acids in a protein with other amino acids having similar characteristics (e.g. charge, side-chain size, hydrophobicity/hydrophilicity, backbone conformation and rigidity, etc.), such that the changes can frequently be made without altering the biological activity of the protein. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4th Ed.)). In addition, substitutions of structurally or functionally similar amino acids are less likely to disrupt biological activity. Exemplary conservative substitutions are listed below:

| Original residue | Conservative substitution |
|---|---|
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

Glycosylation of immunoglobulin G (IgG) in the Fc region, Asn297 (according to the EU numbering system), has been shown to be a requirement for optimal recognition and activation of effector pathways including antibody dependent cellular cytotoxicity (ADCC) and complement dependent cytotoxicity (CDC), Wright & Morrison, Trends in Biotechnology, 15: 26-31 (1997), Tao & Morrison, J. Immunol., 143(8):2595-2601 (1989). As such, glycosylation engineering in the constant region of IgG has become an area of active research for the development of therapeutic monoclonal antibodies (mAbs). It has been established that the presence of N-linked glycosylation at Asn297 is critical for mAb activity in immune effector function assays including ADCC, Rothman (1989), Lifely et al., Glycobiology, 5:813-822 (1995), Umana (1999), Shields (2002), and Shinkawa (2003), and complement dependent cytotoxicity (CDC), Hodoniczky et al., Biotechnol. Prog., 21(6): 1644-1652 (2005), and Jefferis et al., Chem. Immunol., 65: 111-128 (1997). This effect on function has been attributed to the specific conformation adopted by the glycosylated Fc domain, which appears to be lacking when glycosylation is absent. More specifically, IgG which lacks glycosylation in the Fc C_{H}2 domain does not bind to FcγR, including FcγRI, FcγRI, and FcγRIII, Rothman (1989).

Not only does the presence of glycosylation appear to play a role in the effector function of an antibody, the particular composition of the N-linked oligosaccharide is also important. For example, the presence of fucose shows a marked effect on in vitro FcγRIIIa binding and in vitro ADCC, Rothman (1989), and Li et al., Nat. Biotechnol. 24(2): 2100-215 (2006). Recombinant antibodies produced by mammalian cell culture, such as CHO or NSO, contain N-linked oligosaccharides that are predominately fucosylated, Hossler et al., Biotechnology and Bioengineering, 95(5):946-960 (2006), Umana (1999), and Jefferis et al., Biotechnol. Prog. 21:11-16 (2005). Additionally, there is evidence that sialylation in the Fc region may impart anti-inflammatory properties to antibodies. Intravenous immunoglobulin (IVIG) purified over a lectin column to enrich for the sialylated form showed a distinct anti-inflammatory effect limited to the sialylated Fc fragment and was linked to an increase in expression of the inhibitory receptor FcγRIIb, Nimmerjahn and Ravetch., J. Exp. Med. 204:11-15 (2007).

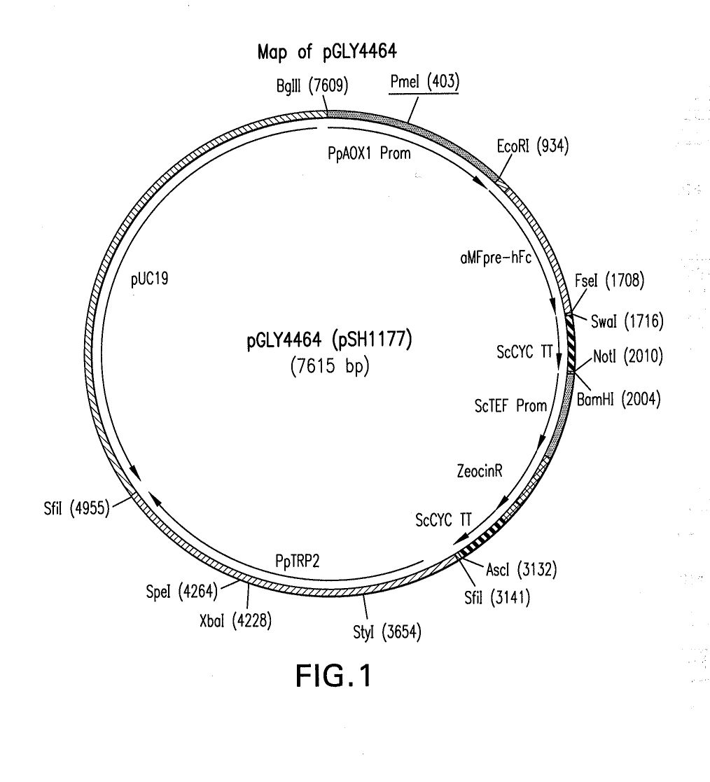

Glycosylation in the Fc region of an antibody derived from mammalian cell lines typically consists of a heterogeneous mix of glycoforms, with the predominant forms typically being comprised of the complex fucosylated glycoforms: G0F, G1F, and, to a lesser extent, G2F. Possible conditions resulting in incomplete galactose transfer to the G0F structure include, but are not limited to, non-optimized galactose transfer machinery, such as β-1,4 galactosyl transferase, and poor UDP-galactose transport into the Golgi apparatus, suboptimal cell culture and protein expression conditions, and steric hindrance by amino acid residues neighboring the oligosaccharide. While each of these conditions may modulate the ultimate degree of terminal galactose, it is thought that subsequent sialic acid transfer to the Fc oligosaccharide is inhibited by the closed pocket configuration of the C_{H}2 domain. See, for example, FIG. 1, Jefferis, R., Nature Biotech., 24 (10): 1230-1231, 2006. Without the correct terminal monosaccharide, specifically galactose, or with insufficient terminal galactosylated forms, there is little possibility of producing a sialylated form, capable of acting as a therapeutic protein, even when produced in the presence of sialyl transferase. Protein engineering and structural analysis of human IgG-Fc glycoforms has shown that glycosylation profiles are affected by Fc conformation, such as the finding that increased levels of galactose and sialic acid on oligosaccharides derived from CHO-produced IgG3 could be achieved when specific amino acids from the Fc pocket were mutated, to an alanine including F241, F243, V264, D265, Y296 and R301. Lund et al., J. Immunol. 157(11); 4963-4969 (1996). It was further shown that certain mutations had some effect on cell mediated superoxide generation and complement mediated red cell lysis, which are used as surrogate markers for FcγRI and C1q binding, respectively.

Yeast have been genetically engineered to produce host strains capable of secreting glycoproteins with highly uniform glycosylation. Choi et al., PNAS, USA 100(9): 5022-5027 (2003) describes the use of libraries of α 1,2 mannosidase catalytic domains and N-acetylglucosaminyltransferase I catalytic domains in combination with a library of fungal type II membrane protein leader sequences to localize the catalytic domains to the secretory pathway. In this way, strains were isolated that produced in vivo glycoproteins with uniform Man₅GlcNAc₂ or GlcNAcMan₅GlcNAc₂ N-glycan structures. Hamilton et al., Science 313 (5792): 1441-1443 (2006) described the production of a glycoprotein, erythropoietin, produced in Pichia pastoris, as having a glycan composition that consisted predominantly of a bisialylated glycan structure, GS6.0, NANA₂Gal₂GlcNAc₂Man₃GlcNAc₂ (90.5%) and monosialylated, GS5.5, NANAGal₂GlcNAc₂ Man₃GlcNAc₂ (7.9%). However, an antibody produced in a similar strain will have a markedly lower content of sialylated N-glycan due to the relatively low level of terminal galactose substrate in the antibody as seen in FIG. 4. It has also recently been shown that sialylation of a Fc oligosaccharide imparts anti-inflammatory properties on therapeutic intravenous gamma globulin and its Fc fragments, Kaneko et al., Science 313(5787): 670-673 (2006), and that the anti-inflammatory activity is dependent on the α-2,6-linked form, but not the α-2,3 form, of sialic acid, Anthony et al., Science, 320: 373-376 (2008).

### Host Organisms and Cell Lines

The Fc-containing polypeptides of this invention can be made in any host organism or cell line. In one embodiment, an Fc-containing polypeptide of the invention is made in a host cell which is capable of producing sialylated N-glycans.

In one embodiment, an Fc-containing polypeptide of the invention is made in a mammalian cell where the cell either endogenously or through genetic or process manipulation produces glycoproteins containing either a mixture of terminal α2-6 and α2-3 sialic acid, or only terminal α2-6 sialic acid. The propagation of mammalian cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture); baby hamster kidney cells (BHK, ATCC CCL 10); Chinese hamster ovary cells/-DHFR (CHO); mouse sertoli cells (TM4); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells; MRC 5 cells; FS4 cells; hybridoma cell lines; NSO; SP2/0; and a human hepatoma line (Hep G2).

In one embodiment, an Fc-containing polypeptide of the invention can be made in a plant cell which is engineered to produce sialylated N-glycans. See, e.g., Cox et al., Nature Biotechnology (2006) 24, 1591-1597 (2006) and Castilho et al., J. Biol. Chem. 285(21): 15923-15930 (2010).

In one embodiment, an Fc-containing polypeptide of the invention can be made in an insect cell which is engineered to produce sialylated N-glycans. See, e.g., Harrison and Jarvis, Adv. Virus Res. 68:159-91 (2006).

In one embodiment, an Fc-containing polypeptide of the invention can be made in a bacterial cell which is engineered to produce sialylated N-glycans. See, e.g., Lizak et al., Bioconjugate Chem. 22:488-496 (2011).

In one embodiment, an Fc-containing polypeptide of the invention can be made in a lower eukaryotic host cell or organism. Recent developments allow the production of fully humanized therapeutics in lower eukaryotic host organisms, yeast and filamentous fungi, such as Pichia pastoris, Gerngross et al., U.S. Pat. No. 7,029,872 and U.S. Pat. No. 7,449,308, the disclosures of which are hereby incorporated by reference. See also Jacobs et al., Nature Protocols 4(1):58-70 (2009).

Due to the decreased FcγR and C1q binding, the materials and methods described herein can be used to produce recombinant glycosylated antibodies with decreased effector function when compared to a parent antibody. Antibodies so produced in Pichia pastoris by the methods of the invention were produced at high yield, with decreased effector function, and had a predominant species of glycoprotein having a terminal α-2,6-linked sialic acid residue as compared to antibodies produced in glycoengineered Pichia pastoris cells lacking the specific Fc mutations or in Pichia pastoris host cells retaining their endogenous glycosylation machinery.

In one embodiment, an Fc-containing polypeptide of the invention is made in a host cell, more preferably a yeast or filamentous fungal host cell, that has been engineered to produce glycoproteins having a predominant N-glycan comprising a terminal sialic acid. In one embodiment of the invention, the predominant N-glycan is the α-2,6 linked form of SA₂Gal₂GlcNAc₂Man₃GlcNAc₂, produced in strains glycoengineered with α-2,6 sialyl transferase which do not produce any α-2,3 linked sialic acid. In other embodiments, the strain will be engineered to express an α-2,3 sialyl transferase alone or in combination with an α-2,6, sialyl transferase, resulting in α-2,3 linked or a combination of α-2,6 and α-2,3 linked sialic acid as the predominant N-glycans.

The cell lines to be used to make the Fc-containing polypeptides of the invention can be any cell line, in particular cell lines with the capability of producing one or more sialylated glycoproteins. Those of ordinary skill in the art would recognize and appreciate that the materials and methods described herein are not limited to the specific strain of Pichia pastoris provided as an example herein, but could include any Pichia pastoris strain or other yeast or filamentous fungal strains in which N-glycans with one or more terminal galactose, such as Gal₂GlcNAc₂Man₃, are produced. The terminal galactose acts as a substrate for the production of α-2,6-linked sialic acid, resulting in the N-glycan structure NANA₂Gal₂GlcNAc₂Man₃GlcNAc₂. Examples of suitable strains are described in U.S. Pat. No. 7,029,872, US 2006-0286637 and Hamilton et al., Science 313 (5792): 1441-1443 (2006), the descriptions of which are incorporated herein as if set forth at length.

In general, lower eukaryotes such as yeast are used for expression of the proteins, particularly glycoproteins because they can be economically cultured, give high yields, and when appropriately modified are capable of suitable glycosylation. Yeast particularly offers established genetics allowing for rapid transformations, tested protein localization strategies and facile gene knock-out techniques. Suitable vectors have expression control sequences, such as promoters, including 3-phosphoglycerate kinase or other glycolytic enzymes, and an origin of replication, termination sequences and the like as desired.

While the invention has been demonstrated herein using the methylotrophic yeast Pichia pastoris, other useful lower eukaryote host cells include Pichia pastoris, Pichia finlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Candida albicans, Aspergillus nidulans, Aspergillus niger, Aspergillus oryzae, Trichoderma reesei, Chrysosporiumi lucknowense, Fusarium sp., Fusarium gramineum, Fusarium venenatum and Neurospora crassa. Various yeasts, such as K. lactis, Pichia pastoris, Pichia methanolica, and Hansenula polymorpha are particularly suitable for cell culture because they are able to grow to high cell densities and secrete large quantities of recombinant protein. Likewise, filamentous fungi, such as Aspergillus niger, Fusarium sp, Neurospora crassa and others can be used to produce glycoproteins of the invention at an industrial scale.

Lower eukaryotes, particularly yeast and filamentous fungi, can be genetically modified so that they express glycoproteins in which the glycosylation pattern is human-like or humanized. As indicated above, the term “human-like N-glycan”, as used herein refers, to the N-linked oligosaccharides which closely resemble the oligosaccharides produced by non-engineered, wild-type human cells. In preferred embodiments of the present invention, the host cells of the present invention are capable of producing human-like glycoproteins with hybrid and/or complex N-glycans; i.e., “human-like N-glycosylation.” The specific “human-like” glycans predominantly present on glycoproteins produced from the host cells of the invention will depend upon the specific engineering steps that are performed. In this manner, glycoprotein compositions can be produced in which a specific desired glycoform is predominant in the composition. Such can be achieved by eliminating selected endogenous glycosylation enzymes and/or genetically engineering the host cells and/or supplying exogenous enzymes to mimic all or part of the mammalian glycosylation pathway as described in U.S. Pat. No. 7,449,308. If desired, additional genetic engineering of the glycosylation can be performed, such that the glycoprotein can be produced with or without core fucosylation. Use of lower eukaryotic host cells is further advantageous in that these cells are able to produce highly homogenous compositions of glycoprotein, such that the predominant glycoform of the glycoprotein may be present as greater than thirty mole percent of the glycoprotein in the composition. In particular aspects, the predominant glycoform may be present in greater than forty mole percent, fifty mole percent, sixty mole percent, seventy mole percent and, most preferably, greater than eighty mole percent of the glycoprotein present in the composition.

Lower eukaryotes, particularly yeast, can be genetically modified so that they express glycoproteins in which the glycosylation pattern is human-like or humanized. Such can be achieved by eliminating selected endogenous glycosylation enzymes and/or supplying exogenous enzymes as described by Gerngross et al., U.S. Pat. No. 7,449,308. For example, a host cell can be selected or engineered to be depleted in α1,6-mannosyl transferase activities, which would otherwise add mannose residues onto the N-glycan on a glycoprotein.

In one embodiment, the host cell further includes an α1,2-mannosidase catalytic domain fused to a cellular targeting signal peptide not normally associated with the catalytic domain and selected to target the α1,2-mannosidase activity to the ER or Golgi apparatus of the host cell. Passage of a recombinant glycoprotein through the ER or Golgi apparatus of the host cell produces a recombinant glycoprotein comprising a Man₅GlcNAc₂ glycoform, for example, a recombinant glycoprotein composition comprising predominantly a Man₅GlcNAc₂ glycoform. For example, U.S. Pat. Nos. 7,029,872 and 7,449,308 and U.S. Published Patent Application No. 2005/0170452 disclose lower eukaryote host cells capable of producing a glycoprotein comprising a Man₅GlcNAc₂ glycoform.

In a further embodiment, the immediately preceding host cell further includes a GlcNAc transferase I (GnT I) catalytic domain fused to a cellular targeting signal peptide not normally associated with the catalytic domain and selected to target GlcNAc transferase I activity to the ER or Golgi apparatus of the host cell. Passage of the recombinant glycoprotein through the ER or Golgi apparatus of the host cell produces a recombinant glycoprotein comprising a GlcNAcMan₅GlcNAc₂ glycoform, for example a recombinant glycoprotein composition comprising predominantly a GlcNAcMan₅GlcNAc₂ glycoform. U.S. Pat. Nos. 7,029,872 and 7,449,308 and U.S. Published Patent Application No. 2005/0170452 disclose lower eukaryote host cells capable of producing a glycoprotein comprising a GlcNAcMan₅GlcNAc₂ glycoform. The glycoprotein produced in the above cells can be treated in vitro with a hexosaminidase to produce a recombinant glycoprotein comprising a Man₅GlcNAc₂ glycoform.

In a further embodiment, the immediately preceding host cell further includes a mannosidase II catalytic domain fused to a cellular targeting signal peptide not normally associated with the catalytic domain and selected to target mannosidase II activity to the ER or Golgi apparatus of the host cell. Passage of the recombinant glycoprotein through the ER or Golgi apparatus of the host cell produces a recombinant glycoprotein comprising a GlcNAcMan₃GlcNAc₂ glycoform, for example a recombinant glycoprotein composition comprising predominantly a GlcNAcMan₃GlcNAc₂ glycoform. U.S. Pat. No. 7,029,872 and U.S. Published Patent Application No. 2004/0230042 discloses lower eukaryote host cells that express mannosidase II enzymes and are capable of producing glycoproteins having predominantly a GlcNAcMan₃GlcNAc₂ glycoform. The glycoprotein produced in the above cells can be treated in vitro with a hexosaminidase to produce a recombinant glycoprotein comprising a Man₃GlcNAc₂ glycoform.

In a further embodiment, the immediately preceding host cell further includes GlcNAc transferase II (GnT II) catalytic domain fused to a cellular targeting signal peptide not normally associated with the catalytic domain and selected to target GlcNAc transferase II activity to the ER or Golgi apparatus of the host cell. Passage of the recombinant glycoprotein through the ER or Golgi apparatus of the host cell produces a recombinant glycoprotein comprising a GlcNAc₂Man₃GlcNAc₂ glycoform, for example a recombinant glycoprotein composition comprising predominantly a GlcNAc₂Man₃GlcNAc₂ glycoform. U.S. Pat. Nos. 7,029,872 and 7,449,308 and U.S. Published Patent Application No. 2005/0170452 disclose lower eukaryote host cells capable of producing a glycoprotein comprising a GlcNAc₂Man₃GlcNAc₂ glycoform. The glycoprotein produced in the above cells can be treated in vitro with a hexosaminidase to produce a recombinant glycoprotein comprising a Man₃GlcNAc₂ glycoform.

In a further embodiment, the immediately preceding host cell further includes a galactosyltransferase catalytic domain fused to a cellular targeting signal peptide not normally associated with the catalytic domain and selected to target galactosyltransferase activity to the ER or Golgi apparatus of the host cell. Passage of the recombinant glycoprotein through the ER or Golgi apparatus of the host cell produces a recombinant glycoprotein comprising a GalGlcNAc₂ Man₃GlcNAc₂ or Gal₂GlcNAc₂Man₃GlcNAc₂ glycoform, or mixture thereof for example a recombinant glycoprotein composition comprising predominantly a GalGlcNAc₂ Man₃GlcNAc₂ glycoform or Gal₂GlcNAc₂Man₃GlcNAc₂ glycoform or mixture thereof. U.S. Pat. No. 7,029,872 and U.S. Published Patent Application No. 2006/0040353 discloses lower eukaryote host cells capable of producing a glycoprotein comprising a Gal₂GlcNAc₂ Man₃GlcNAc₂ glycoform. The glycoprotein produced in the above cells can be treated in vitro with a galactosidase to produce a recombinant glycoprotein comprising a GlcNAc₂Man₃GlcNAc₂ glycoform, for example a recombinant glycoprotein composition comprising predominantly a GlcNAc₂Man₃GlcNAc₂ glycoform.

In a further embodiment, the immediately preceding host cell further includes a sialyltransferase catalytic domain fused to a cellular targeting signal peptide not normally associated with the catalytic domain and selected to target sialyltransferase activity to the ER or Golgi apparatus of the host cell. In a preferred embodiment, the sialyltransferase is an α-2,6-sialyltransferase. Passage of the recombinant glycoprotein through the ER or Golgi apparatus of the host cell produces a recombinant glycoprotein comprising predominantly a NANA₂Gal₂GlcNAc₂Man₃GlcNAc₂ glycoform or NANAGal₂GlcNAc₂Man₃GlcNAc₂ glycoform or mixture thereof. For lower eukaryote host cells such as yeast and filamentous fungi, it is useful that the host cell further include a means for providing CMP-sialic acid for transfer to the N-glycan. U.S. Published Patent Application No. 2005/0260729 discloses a method for genetically engineering lower eukaryotes to have a CMP-sialic acid synthesis pathway and U.S. Published Patent Application No. 2006/0286637 discloses a method for genetically engineering lower eukaryotes to produce sialylated glycoproteins. To enhance the amount of sialylation, it can be advantageous to construct the host cell to include two or more copies of the CMP-sialic acid synthesis pathway or two or more copies of the sialylatransferase. The glycoprotein produced in the above cells can be treated in vitro with a neuraminidase to produce a recombinant glycoprotein comprising predominantly a Gal₂GlcNAc₂Man₃GlcNAc₂ glycoform or GalGlcNAc₂Man₃GlcNAc₂ glycoform or mixture thereof.

Any one of the preceding host cells can further include one or more GlcNAc transferase selected from the group consisting of GnT m, GnT IV, GnT V, GnT VI, and GnT IX to produce glycoproteins having bisected (GnT III) and/or multiantennary (GnT IV, V, VI, and IX) N-glycan structures such as disclosed in U.S. Published Patent Application Nos. 2005/0208617 and 2007/0037248. Further, the proceeding host cells can produce recombinant glycoproteins (for example, antibodies) comprising SA(1-4)Gal(1-4)GlcNAc(2-4) Man₃GlcNAc₂, including antibodies comprising NANA (1-4)Gal(1-4)GlcNAc(2-4) Man3GlcNAc2, NGNA(1-4)Gal(1-4)GlcNAc(2-4)Man3GlcNAc₂ or a combination of NANA (1-4)Gal(1-4)GlcNAc(2-4) Man3GlcNAc₂ and NGNA(1-4)Gal(1-4)GlcNAc(2-4) Man₃GlcNAc₂. In one embodiment, the recombinant glycoprotein will comprise N-glycans comprising a structure selected from the group consisting of SA(1-4)Gal(1-4)GlcNAc(2-4) Man₃GlcNAc₂ and devoid of any α2-3 linked SA.

In further embodiments, the host cell that produces glycoproteins that have predominantly GlcNAcMan₅GlcNAc₂ N-glycans further includes a galactosyltransferase catalytic domain fused to a cellular targeting signal peptide not normally associated with the catalytic domain and selected to target the galactosyltransferase activity to the ER or Golgi apparatus of the host cell. Passage of the recombinant glycoprotein through the ER or Golgi apparatus of the host cell produces a recombinant glycoprotein comprising predominantly the GalGlcNAcMan₅GlcNAc₂ glycoform.

In a further embodiment, the immediately preceding host cell that produced glycoproteins that have predominantly the GalGlcNAcMan₅GlcNAc₂ N-glycans further includes a sialyltransferase catalytic domain fused to a cellular targeting signal peptide not normally associated with the catalytic domain and selected to target sialyltransferase activity to the ER or Golgi apparatus of the host cell. Passage of the recombinant glycoprotein through the ER or Golgi apparatus of the host cell produces a recombinant glycoprotein comprising a SAGalGlcNAcMan₅GlcNAc₂ glycoform (for example NANAGalGlcNAcMan₅GlcNAc₂ or NGNAGalGlcNAcMan₅GlcNAc₂ or a mixture thereof).

Any of the preceding host cells can further include one or more sugar transporters such as UDP-GlcNAc transporters (for example, Kluyveromyces lactis and Mus musculus UDP-GlcNAc transporters), UDP-galactose transporters (for example, Drosophila melanogaster UDP-galactose transporter), and CMP-sialic acid transporter (for example, human sialic acid transporter). Because lower eukaryote host cells such as yeast and filamentous fungi lack the above transporters, it is preferable that lower eukaryote host cells such as yeast and filamentous fungi be genetically engineered to include the above transporters.

Further, any of the preceding host cells can be further manipulated to increase N-glycan occupancy. See e, g., Gaulitzek et al., Biotechnol. Bioengin. 103:1164-1175 (2009); Jones et al., Biochim. Biospvhs. Acta 1726:121-137 (2005); WO2006/107990. In one embodiment, any of the preceding host cells can be further engineered to comprise at least one nucleic acid molecule encoding a heterologous single-subunit oligosaccharyltransferase (for example, Leishmania sp. STT3A protein, STT3B protein, STT3C protein, STT3D protein or combinations thereof) and a nucleic acid molecule encoding the heterologous glycoprotein, and wherein the host cell expresses the endogenous host cell genes encoding the proteins comprising the endogenous OTase complex. In one embodiment, any of the preceding host cells can be further engineered to comprise at least one nucleic acid molecule encoding a Leishmania sp. STT3D protein and a nucleic acid molecule encoding the heterologous glycoprotein, and wherein the host cell expresses the endogenous host cell genes encoding the proteins comprising the endogenous OTase complex.

Host cells further include lower eukaryote cells (e.g., yeast such as Pichia pastoris) that are genetically engineered to produce glycoproteins that do not have α-mannosidase-resistant N-glycans. This can be achieved by deleting or disrupting one or more of the β-mannosyltransferase genes (e.g., BMT1, BMT2, BMT3, and BMT4) (See, U.S. Published Patent Application No. 2006/0211085) and glycoproteins having phosphomannose residues by deleting or disrupting one or both of the phosphomannosyl transferase genes PNO1 and MNN4B (See for example, U.S. Pat. Nos. 7,198,921 and 7,259,007), which in further aspects can also include deleting or disrupting the MNN4A gene. Disruption includes disrupting the open reading frame encoding the particular enzymes or disrupting expression of the open reading frame or abrogating translation of RNAs encoding one or more of the β-mannosyltransferases and/or phosphomannosyltransferases using interfering RNA, antisense RNA, or the like. Further, cells can produce glycoproteins with α-mannosidase-resistant N-glycans through the addition of chemical hinhibios or through modification of the cell culture condition. These host cells can be further modified as described above to produce particular N-glycan structures.

Host cells further include lower eukaryote cells (e.g., yeast such as Pichia pastoris) that are genetically modified to control O-glycosylation of the glycoprotein by deleting or disrupting one or more of the protein O-mannosyltransferase (Dol-P-Man:Protein (Ser/Thr) Mannosyl Transferase genes) (PMTs) (See U.S. Pat. No. 5,714,377) or grown in the presence of Pmtp inhibitors and/or an α-mannosidase as disclosed in Published International Application No. WO 2007/061631, or both. Disruption includes disrupting the open reading frame encoding the Pmtp or disrupting expression of the open reading frame or abrogating translation of RNAs encoding one or more of the Pmtps using interfering RNA, antisense RNA, or the like. The host cells can further include any one of the aforementioned host cells modified to produce particular N-glycan structures.

Pmtp inhibitors include but are not limited to a benzylidene thiazolidinediones. Examples of benzylidene thiazolidinediones that can be used are 5-[[3,4-bis(phenylmethoxy)phenyl]methylene]-4-oxo-2-thioxo-3-thiazolidineacetic Acid; 5-[[3-(1-Phenylethoxy)-4-(2-phenylethoxy)]phenyl]methylene]-4-oxo-2-thioxo-3-thiazolidineacetic Acid; and 5-[[3-(1-Phenyl-2-hydroxy)ethoxy)-4-(2-phenylethoxy)]phenyl]methylene]-4-oxo-2-thioxo-3-thiazolidineacetic acid.

In particular embodiments, the function or expression of at least one endogenous PMT gene is reduced, disrupted, or deleted. For example, in particular embodiments the function or expression of at least one endogenous PMT gene selected from the group consisting of the PMT1, PMT2, PMT3, and PMT4 genes is reduced, disrupted, or deleted; or the host cells are cultivated in the presence of one or more PMT inhibitors. In further embodiments, the host cells include one or more PMT gene deletions or disruptions and the host cells are cultivated in the presence of one or more Pmtp inhibitors. In particular aspects of these embodiments, the host cells also express a secreted α-1,2-mannosidase.

PMT deletions or disruptions and/or Pmtp inhibitors control O-glycosylation by reducing O-glycosylation occupancy, that is, by reducing the total number of O-glycosylation sites on the glycoprotein that are glycosylated. The further addition of an α-1,2-mannsodase that is secreted by the cell controls O-glycosylation by reducing the mannose chain length of the O-glycans that are on the glycoprotein. Thus, combining PMT deletions or disruptions and/or Pmtp inhibitors with expression of a secreted α-1,2-mannosidase controls O-glycosylation by reducing occupancy and chain length. In particular circumstances, the particular combination of PMT deletions or disruptions, Pmtp inhibitors, and α-1,2-mannosidase is determined empirically as particular heterologous glycoproteins (Fabs and antibodies, for example) may be expressed and transported through the Golgi apparatus with different degrees of efficiency and thus may require a particular combination of PMT deletions or disruptions, Pmtp inhibitors, and α-1,2-mannosidase. In another aspect, genes encoding one or more endogenous mannosyltransferase enzymes are deleted. This deletion(s) can be in combination with providing the secreted α-1,2-mannosidase and/or PMT inhibitors or can be in lieu of providing the secreted α-1,2-mannosidase and/or PMT inhibitors.

Thus, the control of O-glycosylation can be useful for producing particular glycoproteins in the host cells disclosed herein in better total yield or in yield of properly assembled glycoprotein. The reduction or elimination of O-glycosylation appears to have a beneficial effect on the assembly and transport of whole antibodies and Fab fragments as they traverse the secretory pathway and are transported to the cell surface. Thus, in cells in which O-glycosylation is controlled, the yield of properly assembled antibodies or Fab fragments is increased over the yield obtained in host cells in which O-glycosylation is not controlled.

To reduce or eliminate the likelihood of N-glycans and O-glycans with β-linked mannose residues, which are resistant to α-mannosidases, the recombinant glycoengineered Pichia pastoris host cells are genetically engineered to eliminate glycoproteins having α-mannosidase-resistant N-glycans by deleting or disrupting one or more of the β-mannosyltransferase genes (e.g., BMT1, BMT2, BMT3, and BMT4) (See, U.S. Pat. No. 7,465,577 and U.S. Pat. No. 7,713,719). The deletion or disruption of BMT2 and one or more of BMT1, BMT3, and BMT4 also reduces or eliminates detectable cross reactivity to antibodies against host cell protein.

Yield of glycoprotein can in some situations be improved by overexpressing nucleic acid molecules encoding mammalian or human chaperone proteins or replacing the genes encoding one or more endogenous chaperone proteins with nucleic acid molecules encoding one or more mammalian or human chaperone proteins. In addition, the expression of mammalian or human chaperone proteins in the host cell also appears to control O-glycosylation in the cell. Thus, further included are the host cells herein wherein the function of at least one endogenous gene encoding a chaperone protein has been reduced or eliminated, and a vector encoding at least one mammalian or human homolog of the chaperone protein is expressed in the host cell. Also included are host cells in which the endogenous host cell chaperones and the mammalian or human chaperone proteins are expressed. In further aspects, the lower eukaryotic host cell is a yeast or filamentous fungi host cell. Examples of the use of chaperones of host cells in which human chaperone proteins are introduced to improve the yield and reduce or control O-glycosylation of recombinant proteins has been disclosed in Published International Application No. WO 2009105357 and WO2010019487 (the disclosures of which are incorporated herein by reference). Like above, further included are lower eukaryotic host cells wherein, in addition to replacing the genes encoding one or more of the endogenous chaperone proteins with nucleic acid molecules encoding one or more mammalian or human chaperone proteins or overexpressing one or more mammalian or human chaperone proteins as described above, the function or expression of at least one endogenous gene encoding a protein O-mannosyltransferase (PMT) protein is reduced, disrupted, or deleted. In particular embodiments, the function of at least one endogenous PMT gene selected from the group consisting of the PMT1, PMT2, PMT3, and PMT4 genes is reduced, disrupted, or deleted.

In addition, O-glycosylation may have an effect on an antibody or Fab fragment's affinity and/or avidity for an antigen. This can be particularly significant when the ultimate host cell for production of the antibody or Fab is not the same as the host cell that was used for selecting the antibody. For example, O-glycosylation might interfere with an antibody's or Fab fragment's affinity for an antigen, thus an antibody or Fab fragment that might otherwise have high affinity for an antigen might not be identified because O-glycosylation may interfere with the ability of the antibody or Fab fragment to bind the antigen. In other cases, an antibody or Fab fragment that has high avidity for an antigen might not be identified because O-glycosylation interferes with the antibody's or Fab fragment's avidity for the antigen. In the preceding two cases, an antibody or Fab fragment that might be particularly effective when produced in a mammalian cell line might not be identified because the host cells for identifying and selecting the antibody or Fab fragment was of another cell type, for example, a yeast or fungal cell (e.g., a Pichia pastoris host cell). It is well known that O-glycosylation in yeast can be significantly different from O-glycosylation in mammalian cells. This is particularly relevant when comparing wild type yeast O-glycosylation with mucin-type or dystroglycan type O-glycosylation in mammals. In particular cases, O-glycosylation might enhance the antibody or Fab fragments affinity or avidity for an antigen instead of interfere with antigen binding. This effect is undesirable when the production host cell is to be different from the host cell used to identify and select the antibody or Fab fragment (for example, identification and selection is done in yeast and the production host is a mammalian cell) because in the production host the O-glycosylation will no longer be of the type that caused the enhanced affinity or avidity for the antigen. Therefore, controlling O-glycosylation can enable use of the materials and methods herein to identify and select antibodies or Fab fragments with specificity for a particular antigen based upon affinity or avidity of the antibody or Fab fragment for the antigen without identification and selection of the antibody or Fab fragment being influenced by the O-glycosylation system of the host cell. Thus, controlling O-glycosylation further enhances the usefulness of yeast or fungal host cells to identify and select antibodies or Fab fragments that will ultimately be produced in a mammalian cell line.

Those of ordinary skill in the art would further appreciate and understand how to utilize the methods and materials described herein in combination with other Pichia pastoris and yeast cell lines that have been genetically engineered to produce specific N-glycans or sialylated glycoproteins, such as, but, not limited to, the host organisms and cell lines described above that have been genetically engineered to produce specific galactosylated or sialylated forms. See, for example, US 2006-0286637, Production of Sialylated N-Glycans in Lower Eukaryotes, in which the pathway for galactose uptake and utilization as a carbon source has been genetically modified, the description of which is incorporated herein as if set forth at length. See also WO2011/149999.

Additionally, the methods herein can be used to produce the above described recombinant Fc-containing polypeptides in other lower eukaryotic cell lines which have been engineered to produce human-like and human glycoproteins that do not have α-2,6 sialyltransferase activity. The methods can also be used to produce the above described recombinant Fc-containing polypeptides in eukaryotic cell lines in which production of sialylated N-glycans is an innate feature.

Levels of α-2,3 and α-2,6 linked sialic acid on the Fc-containing polypeptides can be measured using well known techniques including nuclear magnetic resonance (NMR), normal phase high performance liquid chromatography (HPLC), and high performance anion exchange chromatography with pulsed amperometric detection (HPAEC-PAD).

### Biological Properties of Fc Muteins

For many Fc-containing polypeptides the lack of, or significant decrease in, effector function and increased anti-inflammatory properties would be desirable characteristics. Further it would be desirable that such Fc-containing polypeptides to inhibit FcRn-IgG interactions and induce a rapid decrease of IgG levels. Fc-containing polypeptides having both of these properties would have superior anti-inflammatory properties, and could be used to treat antibody-mediated diseases or to induce clearance of IgG-toxin or IgG-drug complexes.

Vaccaro et al., Nature Biotechnology 23(10) 1283-1288(2005) has demonstrated that mutations at Fc region between CH2 and CH3 domain (Abdeg) result in increased FcRn binding at both pH6 and pH7. Patel et al., J. Immunol. 187; 1015-1022 (2011) was able to show that in a K/BXN model FcRn blockade is a primary contributing factor toward the observed reduction in disease severity.

### Production of Fc-Containing Polypeptides

The Fc-containing polypeptides of the invention can be made according to any method known in the art suitable for generating polypeptides comprising an Fc region having sialylated N-glycans. In one embodiment, the Fc-containing polypeptide is an antibody or an antibody fragment (including, without limitation a polypeptide consisting of or consisting essentially of the Fc region of an antibody). In another embodiment, the Fc-containing polypeptide is an immunoadhesin. Methods of preparing antibody and antibody fragments are well known in the art. Methods of introducing point mutations into a polypeptide, for example site directed mutagenesis, are also well known in the art.

In one embodiment, the Fc-containing polypeptides of the invention are expressed in a host cell that has naturally expresses an α-2,6 sialic acid transferase. In one embodiment, the Fc-containing polypeptides of the invention are expressed in a host cell that has been transformed with a nucleic acid encoding an α-2,6 sialic acid transferase. In one embodiment the host cell is a mammalian cell. In one embodiment, the host cell is a lower eukaryotic host cell. In one embodiment, the host cell is fungal host cell. In one embodiment, the host cell is Pichia sp. In one embodiment, the host cell is Pichia pastoris. In one embodiment, said host cell is capable of producing Fc-polypeptides comprising sialylated N-glycans, wherein the sialic acid residues in the sialylated N-glycans contain alpha-2,6 linkages. In one embodiment, said host cell is capable of producing Fc-containing polypeptides, wherein at least 30%, 40%, 50%, 60%, 70%, 80% or 90% of the N-glycans on the Fc-containing polypeptide comprise an N-linked oligosaccharide structure selected from the group consisting of SA₂Gal₍₁₋₄₎GlcNAc₍₂₋₄₎Man₃GlcNAc₂. In one embodiment, at least 30%, 40%, 50%, 60%, 70%, 80% or 90% of the N-glycans on the Fc-containing polypeptide comprise an N-linked oligosaccharide structure selected from the group consisting of SA₂Gal₂GlcNAc₂Man₃GlcNAc₂. In one embodiment, at least 80% of the N-glycans on the Fc-containing polypeptide comprise an N-linked oligosaccharide structure selected from the group consisting of SA₂Gal₂GlcNAc₂Man₃GlcNAc₂. In any of the above embodiments, the SA could be NANA or NGNA, or an analog or derivative of NANA or NGNA. In one embodiment, at least 30%, 40%, 50%, 60%, 70%, 80% or 90% of the N-glycans on the Fc-containing polypeptide comprise an N-linked oligosaccharide structure selected from the group consisting of NANA₂Gal₂GlcNAc₂Man₃GlcNAc. In one embodiment, the sialic acid residues in the sialylated N-glycans are attached exclusively via α-2,6 linkages.

### N-Glycan Analysis of Fc Muteins

For many glycoproteins, including certain antibodies, sialylation of the terminal N-linked glycan of an IgG Fc region is essential for producing glycoproteins and antibodies that have the correct conformation to impart therapeutic activity. See, for example, Anthony et al., Science, 320: 373-376 (2008), where terminal sialylation was correlated to anti-inflammatory activity for an IVIG preparation. Sialylation requires the presence of a penultimate galactose, upon which the sialyl transferase acts to form the sialylated glycan. Thus, glycoproteins lacking one or more terminal galactose glycoforms cannot produce antibodies having the α 2,6-linked sialic acid composition associated with anti-inflammatory activity.

The N-glycan composition of the antibodies produced herein in glycoengineered Pichia pastoris GFI5.0 and GFI6.0 strains can be analyzed by matrix-assisted laser desorption ionization/time-of-flight (MALDI-TOF) mass spectrometry after release from the antibody with peptide-N-glycosidase F. Released carbohydrate composition can be quantitated by HPLC on an Allentech Prevail carbo (Alltech Associates, Deerfield Ill.) column.

### FcγR and FcRn Binding of Fc Muteins

The FcγR and FcRn binding of Fc muteins can be determined using any method known in the art.

### Biological Targets

Those of ordinary skill in the art would recognize and appreciate that the materials and methods herein could be used to produce any Fc-containing polypeptide for which the characteristics of enhanced anti-inflammatory activity or decreased effector function would be desirable. It should further be noted that there is no restriction as to the type of Fc-containing polypeptide or antibody so produced by the invention. The Fc region of the Fc-containing polypeptide could be from an IgA, IgD, IgE, IgG or IgM. In one embodiment, the Fc region of the Fc-containing polypeptide is from an IgG, including IgG1, IgG2, IgG3 or IgG4. In one embodiment, Fc region of the Fc-containing polypeptide is from an IgG1. In specific embodiments the antibodies or antibody fragments produced by the materials and methods herein can be humanized, chimeric or human antibodies.

In some embodiments, the Fc-containing polypeptide will bind to human FcRn.

In some embodiments, the Fc-containing polypeptides of the invention will bind to a biological target that is involved in inflammation.

In some embodiments, the Fc-containing polypeptide of the invention will bind to a pro-inflammatory cytokine. In some embodiments, the Fc-containing polypeptide of the invention will bind to a molecule selected from the group consisting of: TNF-α, IL-1, IL-2, IL-4, IL-5, IL-6, IL-8, IL-9, IL-10, IL-12, IL-15, IL-17, IL-18, IL-20, IL-21, IL-22, IL-23, IL-23R, IL-25, IL-27, IL-33, CD2, CD4, CD11A, CD14, CD18, CD19, CD23, CD25, CD40, CD40L, CD20, CD52, CD64, CD80, CD147, CD200, CD200R, TSLP, TSLPR, PD-1, PDL1, CTLA4, VLA-4, VEGF, PCSK9, α4β7-integrin, E-selectin, Fact II, ICAM-3, beta2-integrin, IFNγ, C5, CBL, LCAT, CR3, MDL-1, GITR, ADDL, CGRP, TRKA, IGF1R, RANKL, GTC, αBLys, or the receptor for any of the above mentioned molecules. In one embodiment, the Fc-containing polypeptide of the invention will bind to TNF-α. In another embodiment, the Fc-containing polypeptide of the invention will bind to Her2. In another embodiment, the Fc-containing polypeptide of the invention will bind to PCSK9. In another embodiment, the Fc-containing polypeptide of the invention will bind to TNFR. In another embodiment, the Fc-containing polypeptide of the invention will bind to LCAT. In another embodiment, the Fc-containing polypeptide of the invention will bind to TSLP. In another embodiment, the Fc-containing polypeptide of the invention will bind to PD-1. In another embodiment, the Fc-containing polypeptide of the invention will bind to IL-23.

In some embodiments, the Fc-containing polypeptides of the invention will be specific for an antigen selected from autoimmune antigens, allergens, MHC molecules or Rhesus factor D antigen. See, e.g., the antigens listed in Table 1 of WO2010/10910, which is incorporated herein by reference.

### Methods of Increasing Anti-Inflammatory Properties or Decreasing Effector Function/Cytotoxicity

The invention also comprises a method of increasing the anti-inflammatory properties of an Fc-containing polypeptide comprising: selecting a parent Fc-containing polypeptide that is useful in treating an inflammatory condition (for example, an antibody or immunoadhesin that binds to an antigen that is involved in inflammation) and introducing mutations at positions 252, 254, 256, 433, 434, 243 and 264 of the Fc region in the Fc-containing polypeptide, wherein the numbering is according to the EU index as in Kabat, wherein the Fc-containing polypeptide has increased anti-inflammatory properties when compared to the parent Fc-containing polypeptide. In one embodiment, the Fc-containing polypeptide comprises N-glycans, wherein at least 30%, 40%, 50%, 60%, 70%, 80% or 90% of the N-glycans on the Fc-containing polypeptide comprise an N-linked oligosaccharide structure selected from the group consisting of SA₍₁₋₄₎Gal₍₁₋₄₎GlcNAc₍₂₋₄₎Man₃GlcNAc₂. In one embodiment, at least 30%, 40%, 50%, 60%, 70%, 80% or 90% of the N-glycans on the Fc-containing polypeptide comprise a SA₂Gal₂GlcNAc₂Man₃GlcNAc structure. In one embodiment, at least 30%, 40%, 50%, 60%, 70%, 80% or 90% of the N-glycans on the Fc-containing polypeptide comprise a NANA₂Gal₂GlcNAc₂Man₃GlcNAc structure. In one embodiment, the parent Fc-containing polypeptide is an antibody, antibody fragment or immunoadhesin that binds to an antigen that is involved in inflammation. In one embodiment, the parent Fc-containing polypeptide is an antibody, antibody fragment or immunoadhesin that is already marketed or under development for the treatment of an inflammatory conditions. In another embodiment, the parent Fc-containing polypeptide is an antibody selected from the group consisting of: Muromonab-CD3 (anti-CD3 receptor antibody), Abciximab (anti-CD41 7E3 antibody), Rituximab (anti-CD20 antibody), Daclizumab (anti-CD25 antibody), Basiliximab (anti-CD25 antibody), Palivizumab (anti-RSV (respiratory syncytial virus) antibody), Infliximab (anti-TNFα antibody), Trastuzumab (anti-Her2 antibody), Gemtuzumab ozogamicin (anti-CD33 antibody), Alemtuzumab (anti-CD52 antibody), Ibritumomab tiuxeten (anti-CD20 antibody), Adalimumab (anti-TNFα antibody), Omalizumab (anti-IgE antibody), Tositumomab-131I (iodinated derivative of an anti-CD20 antibody), Efalizumab (anti-CD11a antibody), Cetuximab (anti-EGF receptor antibody), Golimumab (anti-TNFα antibody), Bevacizumab (anti VEGF-A antibody), Natalizumab (anti α4 integrin), Efalizumab (anti CD11a), Cetolizumab (anti-TNFα antibody), Tocilizumab (anti-IL-6R), Ustenkinumab (anti IL-12/23), alemtuzumab (anti CD52), and natalizumab (anti α4 integrin), and variants thereof. In another embodiment, the parent Fc-containing polypeptide is an Fc-fusion protein selected from the group consisting of: Arcalyst/rilonacept (IL1R-Fc fusion), Orencia/abatacept (CTLA-4-Fc fusion), Amevive/alefacept (LFA-3-Fc fusion), Anakinra-Fc fusion (IL-1Ra-Fc fusion protein), etanercept (TNFR-Fc fusion protein), FGF-21-Fc fusion protein, GLP-1-Fc fusion protein, RAGE-Fc fusion protein, ActRIIA-Fc fusion protein, ActRIIB-Fc fusion protein, glucagon-Fc fusion protein, oxyntomodulin-Fc-fusion protein, GM-CSF-Fc fusion protein, EPO-Fc fusion protein, Insulin-Fc fusion protein, proinsulin-Fc fusion protein and insulin precursor-Fc fusion protein, and analogs and variants thereof.

The invention also comprises a method of reducing the effector function of an Fc-containing polypeptide, comprising introducing mutations at positions 252, 254, 256, 433, 434, 243 and 264 of a parent Fc-containing polypeptide, wherein the Fc containing polypeptide has decreased effector function when compared to the parent Fc-containing polypeptide, wherein the numbering is according to the EU index as in Kabat. In one embodiment, the Fc-containing polypeptide comprises N-glycans, wherein at least 30%, 40%, 50%, 60%, 70%, 80% or 90% of the N-glycans on the Fc-containing polypeptide comprise an N-linked oligosaccharide structure selected from the group consisting of SA₍₁₋₄₎Gal₍₁₋₄₎GlcNAc₍₂₋₄₎Man3GlcNAc2. In one embodiment, at least 30%, 40%, 50%, 60%, 70%, 80% or 90% of the N-glycans on the Fc-containing polypeptide comprise a SA₂Gal₂GlcNAc₂Man₃GlcNAc structure. In one embodiment, at least 30%, 40%, 50%, 60%, 70%, 80% or 90% of the N-glycans on the Fc-containing polypeptide comprise a NANA₂Gal₂GlcNAc₂Man₃GlcNAc structure. In one embodiment, the Fc-containing polypeptide is an antibody or antigen binding fragment thereof. In one embodiment, the effector function is ADCC. In another embodiment, the effector function is CDC.

The invention also comprises a method of decreasing cytotoxicity of an Fc-containing polypeptide comprising: selecting a parent Fc-containing polypeptide that is useful in treating an inflammatory condition (for example, an antibody or immunoadhesin that binds to an antigen that is involved in inflammation) that binds to an antigen that is involved in inflammation and introducing mutations at positions 252, 254, 256, 433, 434, 243 and 264 of the Fc-containing polypeptide, wherein the numbering is according to the EU index as in Kabat, wherein the Fc-containing polypeptide has decreased cytotoxicity when compared to the parent Fc-containing polypeptide. In one embodiment, the Fc-containing polypeptide comprises N-glycans, wherein at least 30%, 40%, 50%, 60%, 70%, 80% or 90% of the N-glycans on the Fc-containing polypeptide comprise an N-linked oligosaccharide structure selected from the group consisting of SA₍₁₋₄₎Gal₍₁₋₄₎GlcNAc₍₂₋₄₎Man₃GlcNAc₂. In one embodiment, at least 30%, 40%, 50%, 60%, 70%, 80% or 90% of the N-glycans on the Fc-containing polypeptide comprise a SA₂Gal₂GlcNAc₂Man₃GlcNAc structure. In one embodiment, at least 30%, 40%, 50%, 60%, 70%, 80% or 90% of the N-glycans on the Fc-containing polypeptide comprise a NANA₂Gal₂GlcNAc₂Man₃GlcNAc structure.

### Methods of Treatment

The invention also comprises a method of treating an inflammatory condition in a subject in need thereof comprising: administering to the subject a therapeutically effective amount of an Fc-containing polypeptide comprising mutations at positions 252, 254, 256, 433, 434, 243 and 264, wherein the numbering is according to the EU index as in Kabat. In one embodiment, the Fc-containing polypeptide comprises N-glycans, wherein at least 30%, 40%, 50%, 60%, 70%, 80% or 90% of the N-glycans on the Fc-containing polypeptide comprise an N-linked oligosaccharide structure selected from the group consisting of SA₍₁₋₄₎Gal₍₁₋₄₎GlcNAc₍₂₋₄₎Man₃GlcNAc₂. In one embodiment, at least 30%, 40%, 50%, 60%, 70%, 80% or 90% of the N-glycans on the Fc-containing polypeptide comprise a SA₂Gal₂GlcNAc₂Man₃GlcNAc structure. In one embodiment, at least 30%, 40%, 50%, 60%, 70%, 80% or 90% of the N-glycans on the Fc-containing polypeptide comprise a NANA₂Gal₂GlcNAc₂Man₃GlcNAc structure. The Fc-containing polypeptide of the invention can be administered by any route. In one embodiment, the Fc-containing polypeptide is administered parenterally. In one embodiment, the Fc-containing polypeptide is administered subcutaneously.

In one embodiment, the inflammatory condition is unwanted inflammatory immune reactions.

In one embodiment, the inflammatory condition is an autoimmune disease. In one embodiment, the inflammatory condition will be multiple sclerosis. In one embodiment, the inflammatory condition is systemic lupus erythematosus. In one embodiment, the inflammatory condition is type I diabetes.

In one embodiment, the inflammatory condition is a primary immunodeficiency syndrome, including congenital agammaglobulinaemia and hypogammaglobulinaemia, common variable immunodeficiency, severed combined immunodeficiency, or Wiskott Aldrich syndrome.

In one embodiment, the inflammatory condition is a secondary immunodeficiency syndrome, including B-cell lymphocytic leukemia, HIV infection or an allogeneic bone marrow transplantation.

In one embodiment, the inflammatory condition is idiopathic thrombocytopenic purpura.

In one embodiment, the inflammatory condition is multiple myeloma.

In one embodiment, the inflammatory condition is Guillain-Barre syndrome.

In one embodiment, the inflammatory condition is Kawasaki disease.

In one embodiment, the inflammatory condition is chronic inflammatory demyelinating polyneropathy (CIDP).

In one embodiment, the inflammatory condition is autoimmune nuetropenia.

In one embodiment, the inflammatory condition is hemolytic anemia.

In one embodiment, the inflammatory condition is anti-Factor VIII autoimmune disease.

In one embodiment, the inflammatory condition is multifocal neuropathy.

In one embodiment, the inflammatory condition is systemic vasculitis (ANCA positive).

In one embodiment, the inflammatory condition is polymyositis.

In one embodiment, the inflammatory condition is dermatomyositis.

In one embodiment, the inflammatory condition is antiphospholipid syndrome.

In one embodiment, the inflammatory condition is sepsis syndrome.

In one embodiment, the inflammatory condition is graft-v-host disease.

In one embodiment, the inflammatory condition is allergy.

In one embodiment, the inflammatory condition is an anti-Rhesus factor D reaction.

In one embodiment, the inflammatory condition is an inflammatory condition of the cardiovascular system. The Fc-containing polypeptides of the invention may be used to treat atherosclerosis, atherothrombosis, coronary artery hypertension, acute coronary syndrome and heart failure, all of which are associated with inflammation.

In one embodiment, the inflammatory condition is an inflammatory condition of the central nervous system. In another embodiment, the inflammatory condition will be an inflammatory condition of the peripheral nervous system. For example, the Fc-containing polypeptides of the invention may be used for the treatment of, e.g., Alzheimer's disease, amyotrophic lateral sclerosis (a.k.a. ALS; Lou Gehrig's disease), ischemic brain injury, prion diseases, and HIV-associated dementia.

In one embodiment, the inflammatory condition is an inflammatory condition of the gastrointestinal tract. For example, the Fc-containing polypeptides of the invention may be used for treating inflammatory bowel disorders, e.g., Crohn's disease, ulcerative colitis, celiac disease, and irritable bowel syndrome.

In one embodiment, the inflammatory condition is psoriasis, atopic dermatitis, arthritis, including rheumatoid arthritis, osteoarthritis, and psoriatic arthritis.

In one embodiment, the inflammatory condition is steroid-dependent atopic dermatitis.

In one embodiment, the inflammatory condition is cachexia.

Examples of other inflammatory disorders that can be treated using the Fc-containing polypeptides of the invention also include: acne vulgaris, asthma, autoimmune diseases, chronic prostatitis, glomerulonephritis, hypersensitivities, pelvic inflammatory disease, reperfusion injury, sarcoidosis, transplant rejection, vasculitis, interstitial cystitis and myopathies.

In one embodiment, the Fc-containing polypeptide of the invention will be administered a dose of between 1 to 100 milligrams per kilograms of body weight. In one embodiment, the Fc-containing polypeptide of the invention will be administered a dose of between 0.001 to 10 milligrams per kilograms of body weight. In one embodiment, the Fc-containing polypeptide of the invention will be administered a dose of between 0.001 to 0.1 milligrams per kilograms of body weight. In one embodiment, the Fc-containing polypeptide of the invention will be administered a dose of between 0.001 to 0.01 milligrams per kilograms of body weight.

### Pharmaceutical Formulations

The invention also comprises pharmaceutical formulations comprising an Fc-containing polypeptide of the invention and a pharmaceutically acceptable carrier. In one embodiment, the pharmaceutical formulation comprises and Fc-containing polypeptide comprising N-glycans, wherein at least 30%, 40%, 50%, 60%, 70%, 80% or 90% of the N-glycans on the Fc-containing polypeptide comprise an N-linked oligosaccharide structure selected from the group consisting of SA₍₁₋₄₎Gal₍₁₋₄₎GlcNAc₍₂₋₄₎Man₃GlcNAc₂. In one embodiment, at least 30%, 40%, 50%, 60%, 70%, 80% or 90% of the N-glycans on the Fc-containing polypeptide comprise a SA₂Gal₂GlcNAc₂Man₃GlcNAc structure. In one embodiment, at least 30%, 40%, 50%, 60%, 70%, 80% or 90% of the N-glycans on the Fc-containing polypeptide comprise a NANA₂Gal₂GlcNAc₂Man₃GlcNAc structure.

In one embodiment, the invention relates a pharmaceutical composition comprising an Fc-containing polypeptide, wherein at least 70% of the N-glycans on the Fc-containing polypeptide comprise an oligosaccharide structure selected from the group consisting of NANA₍₁₋₄₎Gal₍₁₋₄₎GlcNAc₍₂₋₄₎Man₃GlcNAc₂, wherein the Fc-containing polypeptide comprises mutations at amino acid positions 252, 254, 256, 433, 434, 243 and 264 of the Fc region, wherein the numbering is according to the EU index as in Kabat. In one embodiment, at least 47 mole % of the N-glycans have the structure NANA₂Gal₂GlcNAc₂Man₃GlcNAc₂. In one embodiment, the sialic acid residues in the sialylated N-glycans are attached via an α-2,6 linkage. In one embodiment, the sialic acid residues in the sialylated N-glycans are attached via an α-2,6 linkage and there is no detectable level of an α-2,3 linked sialic acid. In one embodiment, the sialylated N-glycans will comprise no N-glycolylneuraminic acid (NGNA).

As utilized herein, the term “pharmaceutically acceptable” means a non-toxic material that does not interfere with the effectiveness of the biological activity of the active ingredient(s), approved by a regulatory agency of the Federal or a state government or listed in the U.S. Pharmacopoeia or other generally recognized pharmacopoeia for use in animals and, more particularly, in humans. The term “carrier” refers to a diluent, adjuvant, excipient, or vehicle with which the therapeutic is administered and includes, but is not limited to such sterile liquids as water and oils. The characteristics of the carrier will depend on the route of administration.

Pharmaceutical Formulations of therapeutic and diagnostic agents may be prepared by mixing with acceptable carriers, excipients, or stabilizers in the form of, e.g., lyophilized powders, slurries, aqueous solutions or suspensions (see, e.g., Hardman et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, N.Y.).

The mode of administration can vary. Suitable routes of administration include oral, rectal, transmucosal, intestinal, parenteral; intramuscular, subcutaneous, intradermal, intramedullary, intrathecal, direct intraventricular, intravenous, intraperitoneal, intranasal, intraocular, inhalation, insufflation, topical, cutaneous, transdermal, or intra-arterial.

In certain embodiments, the Fc-containing polypeptides of the invention can be administered by an invasive route such as by injection (see above). In some embodiments of the invention, the Fc-containing polypeptides of the invention, or pharmaceutical composition thereof, is administered intravenously, subcutaneously, intramuscularly, intraarterially, intra-articularly (e.g. in arthritis joints), intratumorally, or by inhalation, aerosol delivery. Administration by non-invasive routes (e.g., orally; for example, in a pill, capsule or tablet) is also within the scope of the present invention.

In certain embodiments, the Fc-containing polypeptides of the invention can be administered by an invasive route such as by injection (see above). In some embodiments of the invention, the Fc-containing polypeptides of the invention, or pharmaceutical composition thereof, is administered intravenously, subcutaneously, intramuscularly, intraarterially, intra-articularly (e.g. in arthritis joints), intratumorally, or by inhalation, aerosol delivery. Administration by non-invasive routes (e.g., orally; for example, in a pill, capsule or tablet) is also within the scope of the present invention.

Compositions can be administered with medical devices known in the art. For example, a pharmaceutical composition of the invention can be administered by injection with a hypodermic needle, including, e.g., a prefilled syringe or autoinjector.

The pharmaceutical compositions of the invention may also be administered with a needleless hypodermic injection device; such as the devices disclosed in U.S. Pat. Nos. 6,620,135; 6,096,002; 5,399,163; 5,383,851; 5,312,335; 5,064,413; 4,941,880; 4,790,824 or 4,596,556.

The pharmaceutical compositions of the invention may also be administered by infusion. Examples of well-known implants and modules form administering pharmaceutical compositions include: U.S. Pat. No. 4,487,603, which discloses an implantable micro-infusion pump for dispensing medication at a controlled rate; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments. Many other such implants, delivery systems, and modules are well known to those skilled in the art.

Alternately, one may administer the antibody in a local rather than systemic manner, for example, via injection of the antibody directly into an arthritic joint, often in a depot or sustained release formulation. Furthermore, one may administer the antibody in a targeted drug delivery system, for example, in a liposome coated with a tissue-specific antibody, targeting, for example, arthritic joint or pathogen-induced lesion characterized by immunopathology. The liposomes will be targeted to and taken up selectively by the afflicted tissue.

The administration regimen depends on several factors, including the serum or tissue turnover rate of the therapeutic antibody, the level of symptoms, the immunogenicity of the therapeutic antibody, and the accessibility of the target cells in the biological matrix. Preferably, the administration regimen delivers sufficient therapeutic antibody to effect improvement in the target disease state, while simultaneously minimizing undesired side effects. Accordingly, the amount of biologic delivered depends in part on the particular therapeutic antibody and the severity of the condition being treated. Guidance in selecting appropriate doses of therapeutic antibodies is available (see, e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, N.Y.; Bach (ed.) (1993) Monoclonal Antibodies and Peptide Therapy in Autoimmune Diseases, Marcel Dekker, New York, N.Y.; Baert, et al. (2003) New Engl. J. Med. 348:601-608; Milgrom et al. (1999) New Engl. J. Med. 341:1966-1973; Slamon et al. (2001) New Enl. J. Med. 344:783-792; Beniaminovitz et al. (2000) New Engl. J. Med. 342:613-619; Ghosh et al. (2003) New Engl. J. Med. 348:24-32; Lipsky et al. (2000) New Engl. J. Med. 343:1594-1602).

Determination of the appropriate dose is made by the clinician, e.g., using parameters or factors known or suspected in the art to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced. Preferably, a biologic that will be used is derived from the same species as the animal targeted for treatment, thereby minimizing any immune response to the reagent. In the case of human subjects, for example, chimeric, humanized and fully human Fc-containing polypeptides are preferred.

Fc-containing polypeptides can be provided by continuous infusion, or by doses administered, e.g., daily, 1-7 times per week, weekly, bi-weekly, monthly, bimonthly, quarterly, semiannually, annually etc. Doses may be provided, e.g., intravenously, subcutaneously, topically, orally, nasally, rectally, intramuscular, intracerebrally, intraspinally, or by inhalation. A total weekly dose is generally at least 0.05 μg/kg body weight, more generally at least 0.2 μg/kg, 0.5 μg/kg, 1 μg/kg, 10 μg/kg, 100 μg/kg, 0.25 mg/kg, 1.0 mg/kg, 2.0 mg/kg, 5.0 mg/ml, 10 mg/kg, 25 mg/kg, 50 mg/kg or more (see, e.g., Yang et al., New Engl. J. Med. 349:427-434 (2003); Herold et al., New Engl. J. Med. 346:1692-1698 (2002); Liu et al., J. Neurol. Neurosurg. Psych. 67:451-456 (1999); Portielji et al., Cancer Immunol. Immunother. 52:133-144 (2003). In other embodiments, an Fc-containing polypeptide Of the present invention is administered subcutaneously or intravenously, on a weekly, biweekly, “every 4 weeks,” monthly, bimonthly, or quarterly basis at 10, 20, 50, 80, 100, 200, 500, 1000 or 2500 mg/subject.

As used herein, the terms “therapeutically effective amount”, “therapeutically effective dose” and “effective amount” refer to an amount of an Fc-containing polypeptide of the invention that, when administered alone or in combination with an additional therapeutic agent to a cell, tissue, or subject, is effective to cause a measurable improvement in one or more symptoms of a disease or condition or the progression of such disease or condition. A therapeutically effective dose further refers to that amount of the Fc-containing polypeptide sufficient to result in at least partial amelioration of symptoms, e.g., treatment, healing, prevention or amelioration of the relevant medical condition, or an increase in rate of treatment, healing, prevention or amelioration of such conditions. When applied to an individual active ingredient administered alone, a therapeutically effective dose refers to that ingredient alone. When applied to a combination, a therapeutically effective dose refers to combined amounts of the active ingredients that result in the therapeutic effect, whether administered in combination, serially or simultaneously. An effective amount of a therapeutic will result in an improvement of a diagnostic measure or parameter by at least 10%; usually by at least 20%; preferably at least about 30%; more preferably at least 40%, and most preferably by at least 50%. An effective amount can also result in an improvement in a subjective measure in cases where subjective measures are used to assess disease severity.

### Example 1

### Construction of Expression Constructs of Human IgG1 Fc Variants

DNA encoding human IgG1 Fc variants are chemically synthesized and cloned into pGLY4644 EcoR1 and Fse1 sites (FIG. 1). Final plasmids are named as pGLY11558 through pGLY11565. The sequences of the Fc variants encoded by these plasmids correspond to SEQ ID NOs:1-16.

TABLE 1
List of expression plasmid of Fc variants

| Plasmids | Decription of Fc variants | SEQ ID NO: |
|---|---|---|
| pGLY11558 | F243A, M252Y, S354T, T256E, | 1-2 |
| | H433K, N434F | |
| pGLY11559 | F243Y, V264G, M252Y, S354T, | 3-4 |
| | T256E, H433K, N434F | |
| pGLY11560 | F243L, V264N, M252Y, S354T, | 5-6 |
| | T256E, H433K, N434F | |
| pGLY11561 | F243L, V264A, M252Y, S354T, | 7-8 |
| | T256E, H433K, N434F | |
| pGLY11562 | F243V, V264G, M252Y, S354T, | 9-10 |
| | T256E, H433K, N434F | |
| pGLY11563 | F243A, D265A, M252Y, S354T, | 11-12 |
| | T256E, H433K, N434F | |
| pGLY11564 | V264A, D265A, M252Y, S354T, | 13-14 |
| | T256E, H433K, N434F | |
| pGLY11565 | D265A, R301A, M252Y, S354T, | 15-16 |
| | T256E, H433K, N434F | |
| pGLY11543 | F243A, V264A, M252Y, S354T, | 19-20 |
| | T256E, H433K, N434F | |
| pGLY11546 | M252Y, S354T, T256E, H433K, | 21-22 |
| | N434F | |
| pGLY11533 | F243A, V264 | 23-24 |

### Example 2

### Construction of Expression Constructs of an Anti-TNF Antibody Comprising Mutations in its Fc Region

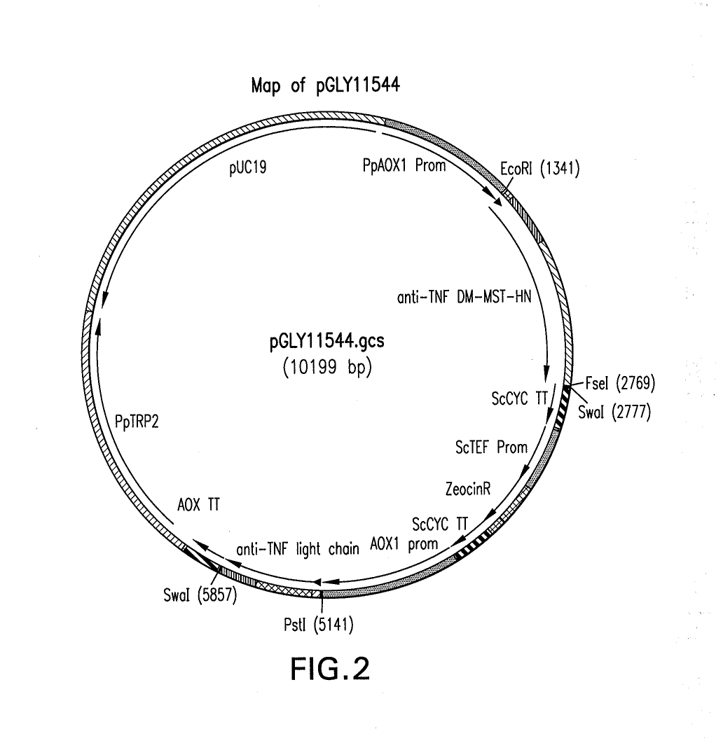

An human antibody IgG1 containing F243A, V264A, M252Y, S354T, T256E, H433K, and N434F at its Fc region is constructed using Fab region binding to human TNF alpha. The heavy chain sequence is provided as SEQ ID NO:17 (designated as “anti-TNF DM-MST-HN” in the plasmid shown in FIG. 2); and the light chain amino acid sequence is provided as SEQ ID NO:18 (designated as “anti-TNF light chain”). The plasmid is named as pGLY11544 and its map is shown in FIG. 2.

For use as controls, the following two human IgG1 antibodies are constructed using the same Fab region binding to human TNF alpha, but comprising the following mutations in the Fc region:
F243A, V264A (antibody comprises the heavy chain of SEQ ID NO:25 and the light chain of SEQ ID NO:18);
M252Y, S354T, T256E, H433K, N434F (antibody comprises the heavy chain of SEQ ID NO:26 and the light chain of SEQ ID NO:18).

### Example 3

### Yeast Transformation and Production of Fc-Containing Polypeptides

The plasmids/nucleic acids described in Examples 1 and 2 can be transformed using routine procedures.

In order to produce Fc-containing polypeptides having α-2,6 sialylated N-glycans, the Pichia pastoris host strain GFI 6.0 YGLY22834 can be used. YGLY22834 is capable of producing proteins with a biantennary N-glycan structure on which terminal α-2,6 linked sialic acid is attached to galactose. The GFI 6.0 YGLY22834 strain has the following genotype: ura5Δ::ScSUC2; och1Δ::lacZ; bmt2Δ::lacZ/KlMNN2-2; mnn4L1Δ::lacZ/MmSLC35A3; pno1Δ mnn4Δ::lacZ; ADE1::lacZ/NA10/MmSLC35A3/FB8; his1Δ::lacZ/ScGAL10/XB33/DmUGT; arg1Δ::HIS1/KD53/TC54; bmt4Δ::lacZ, bmt1Δ::lacZ; bmt3Δ::lacZ; TRP2::ARG1/MmCST/HsGNE/HsCSS/HsSPS/MmST6-33; stel3Δ::lacZ/TrMDS1; dap2Δ::NatR; TRP5::HygRMmCST/HsGNE/HsCSS/HsSPS/MmST6-33, vps 10-1::lacZ-URA5-lacZ pAOX 1-LmSTT3d.

In order to produce Fc-containing polypeptides without α-2,6 sialylated N-glycans, the Pichia pastoris host strain GFI 5.0 YGLY17108 can be used. This strain is capable of producing proteins with biantennary N-glycan structure having predominantly Gal₂GlcNAc₂Man₃GlcNAc₂ N-glycans. The GFI 5.0 YGLY17108 strain has the following genotype: ura5Δ::ScSUC2 och1Δ::lacZ bmt2Δ::lacZ/KlMNN2-2; mnn4L1Δ::lacZ/MmSLC35A3; pno1Δmnn4Δ::lacZ; ADE1::lacZ/NA10/MmSLC35A3/FB8; his1Δ::lacZ/ScGAL10/XB33/DmUGT; arg1Δ::HIS1/KD53/TC54, bmt4Δ::lacZ bmt1::lacZ bmt3::lacZ-URA5-lacZ; PRO 1::ARG1 AOX1-ScMFalphaCiMNS 1; AOX1-LmSTT3D.

The abbreviations used to describe the genotypes are commonly known and understood by those skilled in the art, and include the following abbreviations:
OCH1 Alpha-1,6-mannosyltransferase
KlMNN2-2 K. lactis UDP-GlcNAc transporter
BMT2 Beta-mannose-transfer (beta-mannose elimination)
MNN4L1 MNN4-like 1 (charge elimination)
MmSLC35A3 Mouse homologue of UDP-GlcNAc transporter
PNO1 Phosphomannosylation of N-glycans (charge elimination)
MNN4 Mannosyltransferase (charge elimination)
ScGAL10 UDP-glucose 4-epimerase
XB33 Truncated HsGalT1 fused to ScKRE2 leader
DmUGT UDP-Galactose transporter
KD53 Truncated DmMNSII fused to ScMNN2 leader
TC54 Truncated RnGNTII fused to ScMNN2 leader
NA10 Truncated HsGNTI fused to PpSEC12 leader
FB8 Truncated MmMNS1A fused to ScSEC12 leader
TrMDS1 Secreted T. reseei MNS1
ADE 1 N-succinyl-5-aminoimidazole-4-carboxamide ribotide (SAICAR) synthetase
MmCST Mouse CMP-sialic acid transporter
HsGNE Human UDP-GlcNAc 2-epimerase/N-acetylmannosamine kinase
HsCSS Human CMP-sialic acid synthase
HsSPS Human N-acetylneuraminate-9-phosphate synthase
MmST6-33 Truncated Mouse α-2,6-sailyl transferase fused to ScKRE2 leader
LmSTT3d Catalytic subunit of oligosaccharyltransferase from Leishmania major

The host strain GFI 6.0 YGLY22834 and GFI 5.0 YGLY17108 were constructed using the procedures disclosed in U.S. Pat. Nos. 7,029,872, 7,449,308, 7,863,020; WO2011/06389; and Hamilton et al., Science, 313: 1441-1443 (2006). Following the procedures disclosed in these patents, one can construct vectors that are useful for genetically engineering lower eukaryotic host cells such that they are capable of expressing a desired polypeptide having a desired N-glycoform as the predominant species. These strains were engineered from NRRL11430 (American Type Culture Collection (ATCC), P.O. Box 1549, Manassas, Va. 20108, USA).

Host strains transformed with the nucleic acids encoding the Fc variants of the invention will be fermented and purified using standard methods known to those skilled in the art.

The N-glycan composition of the Fc variants made can be analyzed using MALDI-TOF and HPLC based methods.

MALDI-TOF analysis of glycans can be carried out as described in Choi et al., Proc. Natl. Acad. Sci. USA 100: 5022-5027 (2003) and Hamilton et al., Science 301: 1244-1246 (2003). After the glycoproteins are reduced and carboxymethylated, N-glycans are released by treatment with peptide-N-glycosidase F. The released oligosaccharides are recovered after precipitation of the protein with ethanol. Molecular weights are determined by using a Voyager PRO linear MALDI-TOF (Applied Biosystems) mass spectrometer with delayed extraction according to the manufacturer's instructions.

To quantify the relative amount of each glycoform by HPLC, the N-glycosidase F released glycans are labeled with 2-aminobenzidine (2-AB) and analyzed by HPLC as described in Choi et al., Proc. Natl. Acad. Sci. USA 100: 5022-5027 (2003) and Hamilton et al., Science 313: 1441-1443 (2006).

### Example 4

### FcRn Binding Assays Using Surface Plasmon Resonance

The effect of the Fc muteins described in Examples 1 and 2 on FcRn binding can be determined as described previously described by Vaccaro et al., Nat Biotechnol. 23(10):1283-8 (2005).

### Example 5

### Effect of Fc Variants on the Clearance of Radiolabeled IgG1

The effect of the Fc muteins on the clearance of 1251-labeled wild-type mouse IgG1 (D1.3) is determined as described by Patel et al., J. Immunol. 187(2):1015-22 (2011). Briefly, drinking water is supplemented with 0.1% Lugol 72 h before radiolabeled mouse IgG1 is injected i.p. into BALB/c mice, and radioactivity is monitored at the indicated times by whole-body counting (Atom Lab 100 Dose Calibrator). Seventy-two hours later, mice are i.v. injected with either PBS, 0.5, 1, or 2 mg MST-HN, and whole-body radioactivity is determined at the indicated times.

### Example 6

### FcγR Binding Assay

The effect of the Fc muteins on Fcγ receptor binding assays is determined using the assays described in Shields et al., J. Biol. Chem. 276: 6591-6604 (2001) with minor modifications. High protein binding 96-well plates (Corning Costar, Lowell, Mass.) are coated with 100 μl per well of Fcγ receptor solutions in PBS. FcγRIIIa-V158 and FcγRIIIa-F158 receptors are expressed using P. pastoris as described in Li et al., Nat. Biotech. 24:210-215(2006).

FcγRIIa and FcγRIIb/c are also expressed in glycoengineered Pichia using a similar method as described in Li et al. The FcγRIIa extracellular domain is PCR amplified from human cDNA and cloned into pCR2.1 topo vector. The Fc gamma receptors are cloned into Pichia expression vector using S. cerevisiae alpha Mating Factor prepro domain and under AOX1 promoter.

The DNA sequence of the extracellular domain of the human Fc gamma receptor IIb/c (NP_{—}003992) carrying its C-terminal 9 His-tag is Pichia codon optimized, and designated pAS197 (GeneArt, Germany). For the plasmid construction, the codon-optimized hFcγRIIb/c (AfeI/KpnI) and Saccharomyces cerevisiae αMFprepro (EcoRI/blunt) are cloned into pGLY2219 at EcoRI and KpnI sites.

For FcγRI, the antibody is coated in assay diluent (1% BSA, PBS, 0.05% Tween20) in monomeric form. For all other receptors, the antibody is coated after dimerization with alkaline phosphatase conjugated anti-human IgG F(ab′)2 (Jackson ImmunoResearch, West Grove, Pa.) for one hour at room temperature. FcγRI bound antibody is also detected using the F(ab′)₂ and all plates are quantified by measuring excitation at 340 nm and emission at 465 nm after an 18 hour incubation with SuperPhos (Virolabs, Chantilly, Va.).

### Example 7

### The Effect of the Fc Muteins of the Invention in Collagen-Antibody Induced Arthritis (AIA) Model

MODEL INDUCTION: AIA (Antibody induced arthritis) is induced with a commercial Arthrogen-CIA® arthritogenic monoclonal antibody (purchased from Chondrex) consisting of a cocktail of 5 monoclonal antibodies, clone A2-10 (IgG2a), F10-21 (IgG2a), D8-6 (IgG2a), D1-2G(IgG2b), and D2-112 (IgG2b), that recognize the conserved epitopes on various species of type II collagen.

ANIMALS: 10 week old B10.RIII male mice which are susceptible to arthritis induction without additional of co-stimulatory factors are used. These animals are purchased from Taconic Farms.

CLINICAL SCORING: Paw swelling is measured daily post-induction of arthritis. The severity of the disease was graded on a 0-3 scale per paw as follows: 0, normal; 1, swelling of one digit; 2, swelling of two or more digits; 3, swelling of the entire paw. The maximal clinical score per mouse is 12.

STUDY DESIGN: Arthritis is induced by passive transfer of 3 mg of anti-CII mAb pathogen cocktail IV on day 0.

Groups of Mice are treated subcutaneously with following reagents:

| Reagent | Dose |
|---|---|
| α-2,6 sialylated human IgG1 Fc F243A/V264A mutein | 33 mpk |
| α-2,6 sialylated human IgG1 Fc M252Y, S254T, | 33 mpk |
| T256E, H433K, N434F, F243A and V264A muteins | |
| α-2,6 sialylated human IgG1 Fc M252Y, S254T, | 10 mpk |
| T256E, H433K, N434F, F243A and V264A muteins | |
| α-2,6 sialylated human IgG1 Fc M252Y, S254T, | 5 mpk |
| T256E, H433K, N434F, F243A and V264A muteins | |
| α-2,6 sialylated human IgG1 Fc M252Y, S254T, | 33 mpk |
| T256E, H433K, N434F muteins | |
| α-2,6 sialylated human IgG1 Fc M252Y, S254T, | 10 mpk |
| T256E, H433K, N434F muteins | |
| α-2,6 sialylated human IgG1 Fc M252Y, S254T, | 5 mpk |
| T256E, H433K, N434F muteins | |
| human IgG1 Fc M252Y, S254T, T256E, H433K, | 33 mpk |
| N434F muteins without α-2,6 sialylated glycans | |
| human IgG1 Fc M252Y, S254T, T256E, H433K, | 10 mpk |
| N434F without α-2,6 sialylated glycans | |
| human IgG1 Fc M252Y, S254T, T256E, H433K, | 5 mpk |
| N434F without α-2,6 sialylated glycans | |

An isotype IgG1 antibody is used as a control.

The sample identified as “α-2,6 sialylated human IgG1 Fc F243A/V264A mutein” corresponds to an anti-TNF antibody comprising the amino acid sequence of SEQ ID NO:25/18 produced in GFI6.0 YGLY22834 strain (described in Example 3).

The sample identified as “α-2,6 sialylated human IgG1 Fc M252Y, S254T, T256E, H433K, N434F, F243A and V264A muteins” corresponds to an anti-TNF antibody comprising the amino acid sequence of SEQ ID NO:17/18 produced in GFI6.0 YGLY22834 strain (described in Example 3).

The sample identified as “α-2,6 sialylated human IgG1 Fc M252Y, S254T, T256E, H433K, N434F muteins” corresponds to an anti-TNF antibody comprising the amino acid sequence of SEQ ID NO:26/18 produced in GFI6.0 YGLY22834 strain (described in Example 3).

The sample identified as “human IgG1 Fc M252Y, S254T, T256E, H433K, N434F without α-2,6 sialylated glycans” corresponds to corresponds to an anti-TNF antibody comprising the amino acid sequence of SEQ ID NO: 26/18 produced in GFI5.0 YGLY17108 (described in Example 3).

Additional reagents may be included. For example, antibodies comprising the same anti-TNF Fab region of the antibodies described above, but including any of the Fc muteins described in Example 1 may be constructed and tested.

### Example 8

### Glycan Composition of Anti-TNF Antibodies Comprising Mutations in the Fc Region

Anti-TNF antibodies comprising mutations in the Fc region were made as described in Example 2, and exopressed in Pichia pastoris strains capable of producing polypeptides comprising sialylated N-glycans. The host cell used was GFI 6.0 YGLY28423, a temperature resistant Pichia pastoris strain with an ATT1 gene knockout. This host cell line was engineered from NRRL11430 (American Type Culture Collection (ATCC), P.O. Box 1549, Manassas, Va. 20108, USA) according to the methods described in Hamilton et al., Science, 313: 1441-1443 (2006) and Hamilton US 2006/0286637. YGLY28423 is capable of producing proteins with a biantennary N-glycan structure on which terminal α 2,6-linked sialic acid is attached to galactose. The strain has the following genotype:

ura5Δ::ScSUC2 och1Δ::lacZ bmt2Δ::lacZKlMNN2-2
mnn4L1Δ::lacZ/MmSLC35A3 pno1Δmnn4Δ::lacZ
ADE1::lacZ/NA10/MmSLC35A3/FB8
his1Δ:: lacZ/ScGAL10/XB33/DmUGT
arg1Δ::HIS/KD53/TC54
bmt4Δ::lacZ bmt1Δ::lacZ bmt3Δ::lacZ

### TRP2::ARG1/MmCST/HsGNE/HsCSS/HsSPS/MmST6-33

ste13Δ::lacZ-URA5-lacZ/TrMDS1 dap2Δ::NatR

### TRP5::HygRMmCST/HsGNE/HsCSS/HsSPS/MmST6-33

att1Δ::ScARR3/LmSTT3D

Host cell strain YGLY30184 was constructed by transforming and expressing plasmid pGLY11544 (encoding a human antibody IgG1 containing mutations: F243A, V264A, M252Y, S354T, T256E, H433K, and N434F as described in Example 2) into GFI6.0 host YGLY28423.

To quantify the relative amount of each glycoform by HPLC, glycans were enzymatically released and fluorescently labeled following the protocol provided by Prozyme. Briefly protein samples were denatured in denaturing buffer and then loaded to pre-wet cartridges. The reduced Cysteine residues were alkylated with blocking buffer. The cartridges were washed with washing buffer for 3 times to remove any residual chemicals and buffers. Meanwhile 2 ul PNGase F was added to 10 ul assay buffer. And the cartridges were equilibrated with assay buffer immediately prior to PNGase F digestion. 10 ul PNGase F was added to each cartridge, and the cartridges were briefly spun to settle PNGase F down. The PNGase F digestion proceeded at 50° C. for 30 minutes. After reaction, 20 ul labeling buffer was added to each cartridge and spun to recover released glycans. The glycans were then labeled with instantAB dye and cleaned up in provided cleanup cartridge. The recovered labeled glycans were stored in 50 ul water for HPLC analysis. The results are shown in Table 2.

TABLE 2
Glycan profiles of anti-TNF antibodies

| | Glycan profile | | | |
|---|---|---|---|---|
| Anti-TNF | A2% | A1% | A1H % | Neutral % |
| Heavy chain of SEQ ID | 82 | 10 | 5 | 3 |
| NO: 25 and light chain | | | | |
| of SEQ ID NO: 18 | | | | |
| Heavy chain of SEQ ID | 6 | 9 | 8 | 29(G0 + G1 + |
| NO: 26 and light chain | | | | G2) + 46 (High |
| of SEQ ID NO: 18 | | | | mannose) + |
| | | | | 3 (Man5) |
| Heavy chain of SEQ ID | 76 | 5 | 5 | 14 |
| NO: 17 and light chain | | | | |
| of SEQ ID NO: 18 | | | | |

### Example 9

### FcRn Binding of Anti-TNF Antibodies Comprising Mutations in the Fc Region

The effect of the claimed mutations on FcRn human and mouse FcRn binding was determined. All analyses were performed at 25° C. with a Biacore T100 instrument (GE Healthcare Biosciences). Active flowcell surfaces of a series S CM5 sensor chip (GE Healthcare Biosciences) were immobilized via amine coupling to ˜200RU of recombinant human FcRn (Sino Biological Inc.) diluted to 5 ug/ml in 10 mM Sodium Acetate pH5.0 or recombinant mouse FcRn (R&D Systems) diluted to 5 ug/ml in 10 mM Sodium Acetate pH4.5. A reference flowcell was made in parallel minus the FcRn. Human IgGs serially diluted in running buffer (1×PBS, 0.05% P20, pH6) were flowed over the FcRn surfaces at 30 uL/min for 420 s and allowed to dissociate for 600 s. Two 30 s injections of 2.5 mM NaOH regenerated the surfaces. Binding interactions were repeated in alternate running buffer 1×PBS, 0.05% P20 pH7.5. Data analysis was performed with Biacore T100 Evaluation Software. Double-referenced binding sensograms were fit to the two state model.

The antibody referred to as HUMIRA® was purchased from Abbott.

The other antibodies were obtained as described in Example 7.

The results are shown in Tables 3 and 4.

TABLE 3
Binding to Human FcRn

| | Complex Formation | | Complex Stabilization | | |
|---|---|---|---|---|---|
| Antibody | ka (1/M*s) | kd (1/s) | ka (1/M*s) | kd (1/s) | KD (nM) |
| HUMIRA ® | 5.99E+06 | 3.13E−01 | 8.32E−04 | 1.55E−03 | 33.97 |
| pH 6 | | | | | |
| HUMIRA ® | — | — | — | — | — (no |
| pH 7.5 | | | | | binding |
| | | | | | observed) |
| Heavy chain of SEQ ID NO: 25; | 2.79E+06 | 3.35E−01 | 1.43E−03 | 1.58E−03 | 52.88 |
| light chain of SEQ ID NO: 18 | | | | | |
| pH 6 | | | | | |
| Heavy chain of SEQ ID NO: 25; | — | — | — | — | — (no |
| light chain of SEQ ID NO: 18 | | | | | binding |
| pH 7.5 | | | | | observed) |
| Heavy chain of SEQ ID NO: 26; | 7.78E+06 | 8.70E−03 | 2.10E−03 | 1.30E−03 | 0.42 |
| light chain of SEQ ID NO: 18 | | | | | |
| pH 6 | | | | | |
| Heavy chain of SEQ ID NO: 26; | 1.40E+06 | 2.10E−01 | 7.10E−04 | 2.70E−03 | 122.00 |
| light chain of SEQ ID NO: 18 | | | | | |
| pH 7.5 | | | | | |
| Heavy chain of SEQ ID NO: 17; | 4.00E+06 | 5.00E−03 | 1.00E−03 | 9.00E−04 | 0.59 |
| light chain of SEQ ID NO: 18 | | | | | |
| pH 6 | | | | | |
| Heavy chain of SEQ ID NO: 17; | 5.30E+06 | 7.80E−01 | 5.30E−03 | 2.30E−03 | 0.00 |
| light chain of SEQ ID NO: 18 | | | | | |
| pH 7.5 | | | | | |

TABLE 3
Binding to Mouse FcRn

| | Complex Formation | | Complex Stabilization | | |
|---|---|---|---|---|---|
| Antibody | ka (1/M*s) | kd (1/s) | ka (1/M*s) | kd (1/s) | KD (nM) |
| HUMIRA ® | 1.59E+05 | 1.53E-02 | 1.43E-03 | 1.07E-04 | 6.66 |
| pH 6 | | | | | |
| HUMIRA ® | — | — | — | — | — (no |
| pH 7.5 | | | | | binding |
| | | | | | observed) |
| Heavy chain of SEQ ID NO: 25; | 3.45E+05 | 3.11E−02 | 1.10E−03 | 4.10E−03 | 71.20 |
| light chain of SEQ ID NO: 17 | | | | | |
| pH 6 | | | | | |
| Heavy chain of SEQ ID NO: 25; | — | — | — | — | — (no |
| light chain of SEQ ID NO:17 | | | | | binding |
| pH 7.5 | | | | | observed) |
| Heavy chain of SEQ ID NO: 26; | 1.79E+06 | 8.10E−02 | 3.89E−02 | 2.40E−04 | 0.28 |
| light chain of SEQ ID NO: 17 | | | | | |
| pH 6 | | | | | |
| Heavy chain of SEQ ID NO: 26; | 5.16E+05 | 1.10E−03 | 4.79E−04 | 7.17E−06 | 31.60 |
| light chain of SEQ ID NO: 17 | | | | | |
| pH 7.5 | | | | | |
| Heavy chain of SEQ ID NO: 16; | 2.29E+06 | 5.64E−02 | 2.97E−02 | 2.60E−04 | 0.21 |
| light chain of SEQ ID NO: 17 | | | | | |
| pH 6 | | | | | |
| Heavy chain of SEQ ID NO: 16; | 4.55E+05 | 1.77E−03 | 1.10E−03 | 3.80E−06 | 13.20 |
| light chain of SEQ ID NO: 17 | | | | | |
| pH 7.5 | | | | | |

SEQUENCE LISTING

| SEQ | | |
|---|---|---|
| ID | | |
| NO: | DESCRIPTION | SEQUENCE |
| 1 | DNA sequence | ATGAGATTTCCTTCAATTTTTACTGCTGTTTTATTCGCAGCATCCTCCGC |
| | Human IgG1 Fc | ATTAGCTGCTGAACCAAAGTCTTGTGACAAGACACACACTTGTCCACCAT |
| | mutein | GTCCAGCTCCAGAATTGTTGGGTGGTCCATCCGTTTTTTTGGCTCCACCA |
| | containing | AAGCCAAAGGACACTTTGTACATCACTAGAGAACCAGAGGTTACATGTGT |
| | mutations of | TGTTGTTGACGTTTCTCACGAGGACCCAGAGGTTAAGTTCAACTGGTACG |
| | F243A, M252Y, | TTGACGGTGTTGAAGTTCACAACGCTAAGACTAAGCCAAGAGAAGAGCAG |
| | S354T, T256E, | TACAACTCCACTTACAGAGTTGTTTCCGTTTTGACTGTTTTGCACCAGGA |
| | H433K, N434F | TTGGTTGAACGGTAAAGAATACAAGTGTAAGGTTTCCAACAAGGCTTTGC |
| | with signal | CAGCTCCAATCGAAAAGACTATCTCCAAGGCTAAGGGTCAACCAAGAGAG |
| | sequence of | CCACAGGTTTACACTTTGCCACCATCCAGAGATGAGTTGACTAAGAACCA |
| | alpha mating | GGTTTCCTTGACTTGTTTGGTTAAGGGATTCTACCCATCCGACATTGCTG |
| | factor | TTGAGTGGGAATCTAACGGTCAACCAGAGAACAACTACAAGACTACTCCA |
| | predomain | CCAGTTTTGGATTCTGACGGTTCCTTCTTCTTGTACTCCAAGTTGACTGT |
| | underlined | TGACAAGTCCAGATGGCAACAGGGTAACGTTTTCTCCTGTTCCGTTATGC |
| | | ATGAGGCTTTGAAGTTTCACTACACTCAAAAGTCCTTGTCTTTGTCCCCT |
| | | GGTAAGTAATGA |
| 2 | Amino acid | M R F P S I F T A V L F A A S S A |
| | sequence of | L A A E P K S C D K T H T C P P C |
| | human IgG1 Fc | P A P E L L G G P S V F L A P P K |
| | region | P K D T L Y I T R E P E V T C V V |
| | containing | V D V S H E D P E V K F N W Y V D |
| | mutations of | G V E V H N A K T K P R E E Q Y N |
| | F243A, M252Y, | S T Y R V V S V L T V L H Q D W L |
| | S354T, T256E, | N G K E Y K C K V S N K A L P A P |
| | H433K N434F | I E K T I S K A K G Q P R E P Q V |
| | with signal | Y T L P P S R D E L T K N Q V S L |
| | sequence of | T C L V K G F Y P S D I A V E W E |
| | alpha mating | S N G Q P E N N Y K T T P P V L D |
| | factor | S D G S F F L Y S K L T V D K S R |
| | predomain | W Q Q G N V F S C S V M H E A L K |
| | underlined | F H Y T Q K S L S L S P G K |
| 3 | DNA sequence | ATGAGATTTCCTTCAATTTTTACTGCTGTTTTATTCGCAGCATCCTCCGC |
| | of human | ATTAGCTGCTGAACCAAAGTCTTGTGACAAGACACACACTTGTCCACCAT |
| | IgG1 Fc | GTCCAGCTCCAGAATTGTTGGGTGGTCCATCCGTTTTTTTGTACCCACCA |
| | muteins | AAGCCAAAGGACACTTTGTACATCACTAGAOAACCAGAGCTTACATGTGT |
| | containing | TGTTGGTGACGTTTCTCACGAGGACCCAGAGGTTAAGTTCAACTGGTACG |
| | mutations of | TTGACGGTGTTGAAGTTCACAACGCTAAGACTAAGCCAAGAGAAGAGCAG |
| | F243Y, V264G, | TACAACTCCACTTACAGAGTTGTTTCCGTTTTGACTGTTTTGCACCAGGA |
| | M252Y, S354T, | TTGGTTGAACGGTAAAGAATACAAGTGTAAGGTTTCCAACAAGGCTTTGC |
| | T256E, H433K, | CAGCTCCAATCGAAAAGACTATCTCCAAGGCTAAGGGTCAACCAAGAGAG |
| | N434F with | CCACAGGTTTACACTTTGCCACCATCCAGAGATGAGTTSACTAAGAACCA |
| | signal | GGTTTCCTTGACTTGTTTGGTTAAGGGATTCTACCCATCCGACATTGCTG |
| | sequence of | TTGAGTGGGAATCTAACGGTCAACCAGAGAACAACTACAAGACTACTCCA |
| | alpha mating | CCAGTTTTGGATTCTGACGGTTCCTTCTTCTTGTACTCCAAGTTGACTGT |
| | factor | TGACAAGTCCAGATGGCAACAGGGTAACGTTTTCTCCTGTTCCGTTATGC |
| | predomain | ATGAGGCTTTGAAGTTTCACTACACTCAAAAGTCCTTGTCTTTGTCCCCT |
| | underlined | GGTAAGTAATGAG |
| 4 | Amino acid | M R F P S I F T A V L F A A S S A |
| | sequence of | L A A E P K S C D K T H T C P P C |
| | human IgG1 Fc | P A P E L L G G P S V F L Y P P K |
| | muteins | P K D T L Y I T R E P E V T C V V |
| | containing | G D V S H E D P E V K F N W Y V D |
| | mutations of | G V E V H N A K T K P R E E Q Y N |
| | F243Y, V264G, | S T Y R V V S V L T V L H Q D W L |
| | M252Y, S354T, | N G K E Y K C K V S N K A L P A P |
| | T256E, H433K, | I E K T I S K A K G Q P R E P Q V |
| | N434F with | Y T L P P S R D E L T K N Q V S L |
| | signal | T C L V K G F Y P S D I A V E W E |
| | sequence of | S N G Q P E N N Y K T T P P V L D |
| | alpha mating | S D G S F F L Y S K L T V D K S R |
| | factor | W Q Q G N V F S C S V M H E A L K |
| | predomain | F H Y T Q K S L S L S P G K |
| | underlined | |
| 5 | DNA sequence | ATGAGATTTCCTTCAATTTTTACTGCTGTTTTATTCGCAGCATCCTCCGC |
| | of human IgG1 | ATTAGCTGCTGAACCAAAGTCTTGTGACAAGACACACACTTGTCCACCAT |
| | Fc containing | GTCCAGCTCCAGAATTGTTGGGTGGTCCATCCGTTTTTTTGTTGCCACCA |
| | mutations of | AAGCCAAAGGACACTTTGTACATCACTAGAGAACCAGAGGTTACATGTGT |
| | F243L, V264N, | TGTTAACGACGTTTCTCACGAGGACCCAGAGGTTAAGTTCAACTGGTACG |
| | M252Y, S354T, | TTGACGGTGTTGAAGTTCACAACGCTAAGACTAAGCCAAGAGAAGAGCAG |
| | T256E, H433K, | TACAACTCCACTTACAGAGTTGTTTCCGTTTTGACTCTTTTGCACCAGGA |
| | N434F with | TTGGTTGAACGGTAAAGAATACAAGTGTAAGGTTTCCAACAAGGCTTTGC |
| | signal | CAGCTCCAATCGAAAAGACTATCTCCAAGGCTAAGGGTCAACCAAGAGAG |
| | sequence of | CCACAGGTTTACACTTTGCCACCATCCAGAGATGAGTTGACTAAGAACCA |
| | alpha mating | GGTTTCCTTGACTTGTTTGGTTAAGGGATTCTACCCATCCGACATTGCTG |
| | factor | TTGAGTGGGAATCTAACGGTCAACCAGAGAACAACTACAAGACTACTCCA |
| | predomain | CCAGTTTTGGATTCTGACGGTTCCTTCTTCTTGTACTCCAAGTTGACTGT |
| | underlined | TGACAAGTCCAGATGGCAACAGGGTAACGTTTTCTCCTGTTCCGTTATGC |
| | | ATGAGGCTTTGAAGTTTCACTACACTCAAAAGTCCTTGTCTTTGTCCCCT |
| | | GGTAAGTAATGAGGCCGGCC |
| 6 | Amino acid | M R F P S I F T A V L F A A S S A |
| | sequence of | L A A E P K S C D K T H T C P P C |
| | human IgG1 Fc | P A P E L L G G P S V F L L P P K |
| | containing | P K D T L Y I T R E P E V T C V V |
| | mutations of | N D V S H E D P E V K F N W Y V D |
| | F243L, V264N, | G V E V H N A K T K P R E E Q Y N |
| | M252Y, S354T, | S T Y R V V S V L T V L H Q D W L |
| | T256E, H433K, | N G K E Y K C K V S N K A L P A P |
| | N434F wuth | I E K T I S K A K G Q P R E P Q V |
| | signal | Y T L P P S R D E L T K N Q V S L |
| | sequence of | T C L V K G F Y P S D I A V E W E |
| | alpha mating | S N G Q P E N N Y K T T P P V L D |
| | factor | S D G S F F L Y S K L T V D K S R |
| | predomain | W Q Q G N V F S C S V M H E A L K |
| | underlined | F H Y T Q K S L S L S P G K |
| 7 | DNA sequence | ATGAGATTTCCTTCAATTTTTACTGCTGTTTTATTCGCAGCATCCTCCGC |
| | of human IgG1 | ATTAGCTGCTGAACCAAAGTCTTGTGACAAGACACACACTTGTCCACCAT |
| | Fc carrying | GTCCAGCTCCAGAATTGTTGGGTGGTCCATCCGTTTTTTTGTTGCCACCA |
| | mutations of | AAGCCAAAGGACACTTTGTACATCACTAGAGAACCAGAGGTTACATGTGT |
| | F243L, V264A, | TGTTGCTGACGTTTCTCACGAGGACCCAGAGGTTAAGTTCAACTGGTACG |
| | M252Y, S354T, | TTGACGGTGTTGAAGTTCACAACGCTAAGACTAAGCCAAGAGAAGAGCAG |
| | T256E, H433K, | TACAACTCCACTTACAGAGTTGTTTCCGTTTTGACTGTTTTGCACCAGGA |
| | N434F with | TTGGTTGAACGGTAAAGAATACAAGTGTAAGGTTTCCAACAAGGCTTTGC |
| | signal | CAGCTCCAATCGAAAAGACTATCTCCAAGGCTAAGGGTCAACCAAGAGAG |
| | sequence of | CCACAGGTTTACACTTTGCCACCATCCAGAGATGAGTTGACTAAGAACCA |
| | alpha mating | GGTTTCCTTGACTTGTTTGGTTAAGGGATTCTACCCATCCGACATTGCTG |
| | factor | TTGAGTGGGAATCTAACGGTCAACCAGAGAACAACTACAAGACTACTCCA |
| | predomain | CCAGTTTTGGATTCTGACGGTTCCTTCTTCTTGTACTCCAAGTTGACTGT |
| | underlined | TGACAAGTCCAGATGGCAACAGGGTAACGTTTTCTCCTGTTCCGTTATGC |
| | | ATGAGGCTTTGAAGTTTCACTACACTCAAAAGTCCTTGTCTTTGTCCCCT |
| | | GGTAAGTAATGA |
| 8 | Amino acid | M R F P S I P T A V L F A A S S A |
| | sequence of | L A A E P K S C D K T H T C P P C |
| | human IgG1 Fc | P A P E L L G G P S V F L L P P K |
| | carring | P K D T L Y I T R E P E V T C V V |
| | mutations of | A D V S H E D P E V K F N W Y V D |
| | F243L, V264A, | G V E V H N A K T K P R E E Q Y N |
| | M252Y, S354T, | S T Y R V V S V L T V L H Q D W L |
| | T256E, H433K, | N G K E Y K C K V S N K A L P A P |
| | N434F with | I E K T I S K A K G Q P R E P Q V |
| | signal | Y T L P P S R D E L T K N Q V S L |
| | sequence of | T C L V K G F Y P S D I A V E W E |
| | alpha mating | S N G Q P E N N Y K T T P P V L D |
| | factor | S D G S F F L Y S K L T V D K S R |
| | predomain | W Q Q G N V F S C S V M H E A L K |
| | underlined | F H Y T Q K S L S L S P G K |
| 9 | DNA sequence | ATGAGATTTCCTTCAATTTTTACTGCTGTTTTATTCGCAGCATCCTCCGC |
| | coding human | ATTAGCTGCTGAACCAAAGTCTTGTGACAAGACACACACTTGTCCACCAT |
| | IgG1 Fc | GTCCAGCTCCAGAATTGTTGGGTGGTCCATCCGTTTTTTTGGTTCCACCA |
| | containing | AAGCCAAAGGACACTTTGTACATCACTAGAGAACCAGAGGTTACATGTGT |
| | mutations of | TGTTGGTGACGTTTCTCACGAGGACCCAGAGGTTAAGTTCAACTGGTACG |
| | F243V, V264G, | TTGACGGTGTTGAAGTTCACAACGCTAAGACTAAGCCAAGAGAAGAGCAG |
| | M252Y, S354T, | TACAACTCCACTTACAGAGTTGTTTCCGTTTTGACTGTTTTGCACCAGGA |
| | T256E, H433K, | TTGGTTGAACGGTAAAGAATACAAGTGTAAGGTTTCCAACAAGGCTTTGC |
| | N434F with | CAGCTCCAATCGAAAAGACTATCTCCAAGGCTAAGGGTCAACCAAGAGAG |
| | signal | CCACAGGTTTACACTTTGCCACCATCCAGAGATGAGTTGACTAAGAACCA |
| | sequence of | GGTTTCCTTGACTTGTTTGGTTAAGGGATTCTACCCATCCGACATTGCTG |
| | alpha mating | TTGAGTGGGAATCTAACGGTCAACCAGAGAACAACTACAAGACTACTCCA |
| | factor | CCAGTTTTGGATTCTGACGGTTCCTTCTTCTTGTACTCCAAGTTGACTGT |
| | predomain | TGACAAGTCCAGATGGCAACAGGGTAACGTTTTCTCCTGTTCCGTTATGC |
| | underlined | ATGAGGCTTTGAAGTTTCACTACACTCAAAAGTCCTTGTCTTTGTCCCCT |
| | | GGTAAGTAATGA |
| 10 | Amino acid | M R F P S I F T A V L F A A S S A |
| | sequence of | L A A E P K S C D K T H T C P P C |
| | human IgG1 Fc | P A P E L L G G P S V F L V P P K |
| | containing | P K D T L Y I T R E P E V T C V V |
| | mutations of | G D V S H E D P E V K F N W Y V D |
| | F243V, V264G, | G V E V H N A K T K P R E E Q Y N |
| | M252Y, S354T, | S T Y R V V S V L T V L H Q D W L |
| | T256E, H433K, | N G K E Y K C K V S N K A L P A P |
| | N434F with | I E K T I S K A K G Q P R E P Q V |
| | signal | Y T L P P S R D E L T K N Q V S L |
| | sequence of | T C L V K G F Y P S D I A V E W E |
| | alpha mating | S N G Q P E N N Y K T T P P V L D |
| | factor | S D G S F F L Y S K L T V D K S R |
| | predomain | W Q Q G N V F S C S V M H E A L K |
| | underlined | F H Y T Q K S L S L S P G K |
| 11 | DNA sequence | ATGAGATTTCCTTCAATTTTTACTGCTGTTTTATTCGCAGCATCCTCCGC |
| | coding human | ATTAGCTGCTGAACCAAAGTCTTGTGACAAGACACACACTTGTCCACCAT |
| | IgG1 Fc | GTCCAGCTCCAGAATTGTTGGGTGGTCCATCCGTTTTTTTGGCTCCACCA |
| | containing | AAGCCAAAGGACACTTTGTACATCACTAGAGAACCAGAGGTTACATGTGT |
| | mutations of | TGTTGTTGCTGTTTCTCACGAGGACCCAGAGGTTAAGTTCAACTGGTACG |
| | F243A, D265A, | TTGACGGTGTTGAAGTTCACAACGCTAAGACTAAGCCAAGAGAAGAGCAG |
| | M252Y, S354T, | TACAACTCCACTTACAGAGTTGTTTCCGTTTTGACTGTTTTGCACCAGGA |
| | T256E, H433K, | TTGGTTGAACGGTAAAGAATACAAGTGTAAGGTTTCCAACAAGGCTTTGC |
| | N434F with | CAGCTCCAATCGAAAAGACTATCTCCAAGGCTAAGGGTCAACCAAGAGAG |
| | signal | CCACAGGTTTACACTTTGCCACCATCCAGAGATGAGTTGACTAAGAACCA |
| | sequence of | GGTTTCCTTGACTTGTTTGGTTAAGGGATTCTACCCATCCGACATTGCTG |
| | alpha mating | TTGAGTGGGAATCTAACGGTCAACCAGAGAACAACTACAAGACTACTCCA |
| | factor | CCAGTTTTGGATTCTGACGGTTCCTTCTTCTTGTACTCCAAGTTGACTGT |
| | predomain | TGACAAGTCCAGATGGCAACAGGGTAACGTTTTCTCCTGTTCCGTTATGC |
| | underlined | ATGAGGCTTTGAAGTTTCACTACACTCAAAAGTCCTTGTCTTTGTCCCCT |
| | | GGTAAGTAATGA |
| 12 | Amino acid | M R F P S I F T A V L F A A S S A |
| | sequence of | L A A E P K S C D K T H T C P P C |
| | human IgG1 Fc | P A P E L L G G P S V F L A P P K |
| | containing | P K D T L Y I T R E P E V T C V V |
| | mutations of | V A V S H E D P E V K F N W Y V D |
| | F243A, D265A, | G V E V H N A K T K P R E E Q Y N |
| | M252Y, S354T, | S T Y R V V S V L T V L H Q D W L |
| | T256E, H433K, | N G K E Y K C K V S N K A L P A P |
| | N434F with | I E K T I S K A K G Q P R E P Q V |
| | signal | Y T L P P S R D E L T K N Q V S L |
| | sequence of | T C L V K G F Y P S D I A V E W E |
| | alpha mating | S N G Q P E N N Y K T T P P V L D |
| | factor | S D G S F F L Y S K L T V D K S R |
| | predomain | W Q Q G N V F S C S V M H E A L K |
| | underlined | F H Y T Q K S L S L S P G K |
| 13 | DNA sequence | ATGAGATTTCCTTCAATTTTTACTGCTGTTTTATTCGCAGCATCCTCCGC |
| | coding human | ATTAGCTGCTGAACCAAAGTCTTGTGACAAGACACACACTTGTCCACCAT |
| | IgG1 Fc | GTCCAGCTCCAGAATTGTTGGGTGGTCCATCCGTTTTTTTGTTTCCACCA |
| | containing | AAGCCAAAGGACACTTTGTACATCACTAGAGAACCAGAGGTTACATGTGT |
| | mutations of | TGTTGCCGCTGTTTCTCACGAGGACCCAGAGGTTAAGTTCAACTGGTACG |
| | V264A, D265A, | TTGACGGTGTTGAAGTTCACAACGCTAAGACTAAGCCAAGAGAAGAGCAG |
| | M252Y, S354T, | TACAACTCCACTTACAGAGTTGTTTCCGTTTTGACTGTTTTGCACCAGGA |
| | T256E, H433K, | TTGGTTGAACGGTAAAGAATACAAGTGTAAGGTTTCCAACAAGGCTTTGC |
| | N434F with | CAGCTCCAATCGAAAAGACTATCTCCAAGGCTAAGGGTCAACCAAGAGAG |
| | signal | CCACAGGTTTACACTTTGCCACCATCCAGAGATGAGTTGACTAAGAACCA |
| | sequence of | GGTTTCCTTGACTTGTTTGGTTAAGGGATTCTACCCATCCGACATTGCTG |
| | alpha mating | TTGAGTGGGAATCTAACGGTCAACCAGAGAACAACTACAAGACTACTCCA |
| | factor | CCAGTTTTGGATTCTGACGGTTCCTTCTTCTTGTACTCCAAGTTGACTGT |
| | predomain | TGACAAGTCCAGATGGCAACAGGGTAACGTTTTCTCCTGTTCCGTTATGC |
| | underlined | ATGAGGCTTTGAAGTTTCACTACACTCAAAAGTCCTTGTCTTTGTCCCCT |
| | | GGTAAGTAATGA |
| 14 | Amino acid | M R F P S I F T A V L F A A S S A |
| | sequence of | L A A E P K S C D K T H T C P P C |
| | human IgG1 Fc | P A P E L L G G P S V F L F P P K |
| | containing | P K D T L Y I T R E P E V T C V V |
| | mutations of | A A V S H E D P E V K F N W Y V D |
| | F243A, D265A, | G V E V H N A K T K P R E E Q Y N |
| | M252Y, S354T, | S T Y R V V S V L T V L H O D W L |
| | T256E, H433K, | N G K E Y K C K V S N K A L P A P |
| | N434F with | I E K T I S K A K G Q P R E P Q V |
| | signal | Y T L P P S R D E L T K N Q V S L |
| | sequence of | T C L V K G F Y P S D I A V E W E |
| | alpha mating | S N G Q P E N N Y K T T P P V L D |
| | factor | S D G S F F L Y S K L T V D K S R |
| | predomain | W Q Q G N V F S C S V M H E A L K |
| | underlined | F H Y T Q K S L S L S P G K |
| 15 | DNA sequence | ATGAGATTTCCTTCAATTTTTACTGCTGTTTTATTCGCAGCATCCTCCGC |
| | coding human | ATTAGCTGCTGAACCAAAGTCTTGTGACAAGACACACACTTGTCCACCAT |
| | IgG1 Fc | GTCCAGCTCCAGAATTGTTGGGTGGTCCATCCGTTTTTTTGTTTCCACCA |
| | containing | AAGCCAAAGGACACTTTGTACATCACTAGAGAACCAGAGGTTACATGTGT |
| | mutations of | TGTTGTTGCTGTTTCTCACGAGGACCCAGAGGTTAAGTTCAACTGGTACG |
| | D265A, R301A, | TTGACGGTGTTGAAGTTCACAACGCTAAGACTAAGCCAAGAGAAGAGCAG |
| | M252Y, S354T, | TACAACTCCACTTACGCTGTTGTTTCCGTTTTGACTGTTTTGCACCAGGA |
| | T256E, H433K, | TTGGTTGAACGGTAAAGAATACAAGTGTAAGGTTTCCAACAAGGCTTTGC |
| | N434F with | CAGCTCCAATCGAAAAGACTATCTCCAAGGCTAAGGGTCAACCAAGAGAG |
| | signal | CCACAGGTTTACACTTTGCCACCATCCAGAGATGAGTTGACTAAGAACCA |
| | sequence of | GGTTTCCTTGACTTGTTTGGTTAAGGGATTCTACCCATCCGACATTGCTG |
| | alpha mating | TTGAGTGGGAATCTAACGGTCAACCAGAGAACAACTACAAGACTACTCCA |
| | factor | CCAGTTTTGGATTCTGACGGTTCCTTCTTCTTGTACTCCAAGTTGACTGT |
| | predomain | TGACAAGTCCAGATGGCAACAGGGTAACGTTTTCTCCTGTTCCGTTATGC |
| | underlined | ATGAGGCTTTGAAGTTTCACTACACTCAAAAGTCCTTGTCTTTGTCCCCT |
| | | GGTAAGTAATGA |
| 16 | Amino acid | M R F P S I F T A V L F A A S S A |
| | sequence of | L A A E P K S C D K T H T C P P C |
| | human IgG1 Fc | P A P E L L G G P S V F L F P P K |
| | containing | P K D T L Y I T R E P E V T C V V |
| | mutations of | V A V S H E D P E V K F N W Y V D |
| | D265A, R301A, | G V E V H N A K T K P R E E Q Y N |
| | M252Y, S354T, | S T Y A V V S V L T V L H Q D W L |
| | T256E, H433K, | N G K E Y K C K V S N K A L P A P |
| | N434F with | I E K T I S K A K G Q P R E P Q V |
| | signal | Y T L P P S R D E L T K N Q V S L |
| | sequence of | T C L V K G F Y P S D I A V E W E |
| | alpha mating | S N G Q P E N N Y K T T P P V L D |
| | factor | S D G S F F L Y S K L T V D K S R |
| | predomain | W Q Q G N V F S C S V M H E A L K |
| | underlined | F H Y T Q K S L S L S P G K |
| 17 | Heavy chain | E V Q L V E S G G G L V Q P G R S |
| | amino acid | L R L S C A A S G F T F D D Y A M |
| | sequence of | H W V R Q A P G K G L E W V S A I |
| | anti-TNF IgG1 | T W N S G H I D Y A D S V E G R F |
| | antibody | T I S R D N A K N S L Y L Q M N S |
| | comprising | L R A E D T A V Y Y C A K V S Y L |
| | mutations at | S T A S S L D Y W G Q G T L V T V |
| | positions | S S A S T K G P S V F P L A P S S |
| | M252Y, S254T, | K S T S G G T A A L G C L V K D Y |
| | T256E, H433K, | F P E P V T V S W N S G A L T S G |
| | N434F, F243A | V H T F P A V L Q S S G L Y S L S |
| | and V264A | S V V T V P S S S L G T Q T Y I C |
| | | N V N H K P S N T K V D K K V E P |
| | | K S C D K T H T C P P C P A P E L |
| | | L G G P S V F L A P P K P K D T L |
| | | Y I T R E P E V T C V V A D V S H |
| | | E D P E V K F N W Y V D G V E V H |
| | | N A K T K P R E E Q Y N S T Y R V |
| | | V S V L T V L H O D W L N G K E Y |
| | | K C K V S N K A L P A P I E K T I |
| | | S K A K G Q P R E P Q V Y T L P P |
| | | S R D E L T K N Q V S L T C L V K |
| | | G F Y P S D I A V E W E S N G Q P |
| | | E N N Y K T T P P V L D S D G S F |
| | | F L Y S K L T V D K S R W Q Q G N |
| | | V F S C S V M H E A L K F H Y T Q |
| | | K S L S L S P G K |
| 18 | Light chain | D I Q M T Q S P S S L S A S V G D |
| | amino acid | R V T I T C R A S Q G I R N Y L A |
| | sequence of | W Y Q Q K P G K A P K L L I Y A A |
| | anti-TNF IgG1 | S T L Q S G V P S R F S G S G S G |
| | antibody | T D F T L T I S S L Q P E D V A T |
| | | Y Y C Q R Y N R A P Y T F G Q G T |
| | | K V E I K R T V A A P S V F I F P |
| | | P S D E Q L K S G T A S V V C L L |
| | | N N F Y P R E A K V Q W K V D N A |
| | | L Q S G N S Q E S V T E Q D S K D |
| | | S T Y S L S S T L T L S K A D Y E |
| | | K H K V Y A C E V T H Q G L S S P |
| | | V T K S F N R G E C |
| 19 | DNA sequence | GCTGAACCAAAGTCTTGTGACAAGACACACACTTGTCCACCATGTCCAGC |
| | of human IgG1 | TCCAGAATTGTTGGGTGGTCCATCCGTTTTTTTGGCTCCACCAAAGCCAA |
| | Fc containing | AGGACACTTTGtacATCactAGAgaaCCAGAGGTTACATGTGTTGTTGCT |
| | mutations | GACGTTTCTCACGAGGACCCAGAGGTTAAGTTCAACTGGTACGTTGACGG |
| | F243A, | TGTTGAAGTTCACAACGCTAAGACTAAGCCAAGAGAAGAGCAGTACAACT |
| | V264A,M252Y, | CCACTTACAGAGTTGTTTCCGTTTTGACTGTTTTGCACCAGGATTGGTTG |
| | S354T, T256E, | AACGGTAAAGAATACAAGTGTAAGGTTTCCAACAAGGCTTTGCCAGCTCC |
| | H433K, N434F | AATCGAAAAGACTATCTCCAAGGCTAAGGGTCAACCAAGAGAGCCACAGG |
| | | TTTACACTTTGCCACCATCCAGAGATGAGTTGACTAAGAACCAGGTTTCC |
| | | TTGACTTGTTTGGTTAAGGGATTCTACCCATCCGACATTGCTGTTGAGTG |
| | | GGAATCTAACGGTCAACCAGAGAACAACTACAAGACTACTCCACCAGTTT |
| | | TGGATTCTGACGGTTCCTTCTTCTTGTACTCCAAGTTGACTGTTGACAAG |
| | | TCCAGATGGCAACAGGGTAACGTTTTCTCCTGTTCCGTTATGCATGAGGC |
| | | TTTGaagtttCACTACACTCAAAAGTCCTTGTCTTTGTCCCCTGGTAAG |
| 20 | Amino acid | A E P K S C D K T H T C P P C P A |
| | sequence of | P E L L G G P S V F L A P P K P K |
| | human IgG1 Fc | D T L Y I T R E P E V T C V V A D |
| | containing | V S H E D P E V K F N W Y V D G V |
| | mutations | E V H N A K T K P R E E Q Y N S T |
| | F243A, | Y R V V S V L T V L H Q D W L N G |
| | V264A,M252Y, | K E Y K C K V S N K A L P A P I E |
| | S354T, T256E, | K T I S K A K G Q P R E P Q V Y T |
| | H433K, N434F | L P P S R D E L T K N Q V S L T C |
| | | L V K G F Y P S D I A V E W E S N |
| | | G Q P E N N Y K T T P P V L D S D |
| | | G S F F L Y S K L T V D K S R W Q |
| | | Q G N V F S C S V M H E A L K F H |
| | | Y T Q K S L S L S P G K |
| 21 | DNA sequence | GCTGAACCAAAGTCTTGTGACAAGACACACACTTGTCCACCATGTCCAGC |
| | of human IgG1 | TCCAGAATTGTTGGGTGGTCCATCCGTTTTTTTGtttCCACCAAAGCCAA |
| | Fc containing | AGGACACTTTGtacATCactAGAgaaCCAGAGGTTACATGTGTTGTTgtt |
| | mutations | GACGTTTCTCACGAGGACCCAGAGGTTAAGTTCAACTGGTACGTTGACGG |
| | M252Y, S354T, | TGTTGAAGTTCACAACGCTAAGACTAAGCCAAGAGAAGAGCAGTACAACT |
| | T256E, H433K, | CCACTTACAGAGTTGTTTCCGTTTTGACTGTTTTGCACCAGGATTGGTTG |
| | N434F | AACGGTAAAGAATACAAGTGTAAGGTTTCCAACAAGGCTTTGCCAGCTCC |
| | | AATCGAAAAGACTATCTCCAAGGCTAAGGGTCAACCAAGAGAGCCACAGG |
| | | TTTACACTTTGCCACCATCCAGAGATGAGTTGACTAAGAACCAGGTTTCC |
| | | TTGACTTGTTTGGTTAAGGGATTCTACCCATCCGACATTGCTGTTGAGTG |
| | | GGAATCTAACGGTCAACCAGAGAACAACTACAAGACTACTCCACCAGTTT |
| | | TGGATTCTGACGGTTCCTTCTTCTTGTACTCCAAGTTGACTGTTGACAAG |
| | | TCCAGATGGCAACAGGGTAACGTTTTCTCCTGTTCCGTTATGCATGAGGC |
| | | TTTGaagtttCACTACACTCAAAAGTCCTTGTCTTTGTCCCCTGGTAAG |
| 22 | Amino acid | A E P K S C D K T H T C P P C P A |
| | sequence of | P E L L G G P S V F L F P P K P K |
| | human IgG1 Fc | D T L Y I T R E P E V T C V V V D |
| | containing | V S H E D P E V K F N W Y V D G V |
| | mutations | E V H N A K T K P R E E Q Y N S T |
| | M252Y, S354T, | Y R V V S V L T V L H Q D W L N G |
| | T256E, H433K, | K E Y K C K V S N K A L P A P I E |
| | N434F | K T I S K A K G Q P R E P Q V Y T |
| | | L P P S R D E L T K N Q V S L T C |
| | | L V K G F Y P S D I A V E W E S N |
| | | G Q P E N N Y K T T P P V L D S D |
| | | G S F F L Y S K L T V D K S R W Q |
| | | Q G N V F S C S V M H E A L K F H |
| | | Y T Q K S L S L S P G K |
| 23 | DNA sequence | GCTGAACCAAAGTCTTGTGACAAGACACACACTTGTCCACCATGTCCAGC |
| | of human IgG1 | TCCAGAATTGTTGGGTGGTCCATCCGTTTTTTTGGCTCCACCAAAGCCAA |
| | Fc containing | AGGACACTTTGATGATCTCCAGAACTCCAGAGGTTACATGTGTTGTTGCT |
| | mutations | GACGTTTCTCACGAGGACCCAGAGGTTAAGTTCAACTGGTACGTTGACGG |
| | F243A, V264A | TGTTGAAGTTCACAACGCTAAGACTAAGCCAAGAGAAGAGCAGTACAACT |
| | | CCACTTACAGAGTTGTTTCCGTTTTGACTGTTTTGCACCAGGATTGGTTG |
| | | AACGGTAAAGAATACAAGTGTAAGGTTTCCAACAAGGCTTTGCCAGCTCC |
| | | AATCGAAAAGACTATCTCCAAGGCTAAGGGTCAACCAAGAGAGCCACAGG |
| | | TTTACACTTTGCCACCATCCAGAGATGAGTTGACTAAGAACCAGGTTTCC |
| | | TTGACTTGTTTGGTTAAGGGATTCTACCCATCCGACATTGCTGTTGAGTG |
| | | GGAATCTAACGGTCAACCAGAGAACAACTACAAGACTACTCCACCAGTTT |
| | | TGGATTCTGACGGTTCCTTCTTCTTGTACTCCAAGTTGACTGTTGACAAG |
| | | TCCAGATGGCAACAGGGTAACGTTTTCTCCTGTTCCGTTATGCATGAGGC |
| | | TTTGCACAACCACTACACTCAAAAGTCCTTGTCTTTGTCCCCTGGTAAG |
| 24 | Amino acid | A E P K S C D K T H T C P P C P A |
| | sequence of | P E L L G G P S V F L A P P K P K |
| | human IgG1 Fc | D T L M I S R T P E V T C V V A D |
| | containing | V S H E D P E V K F N W Y V D G V |
| | mutations | E V H N A K T K P R E E Q Y N S T |
| | F243A, V264A | Y R V V S V L T V L H Q D W L N G |
| | | K E Y K C K V S N K A L P A P I E |
| | | K T I S K A K G Q P R E P Q V Y T |
| | | L P P S R D E L T K N Q V S L T C |
| | | L V K G F Y P S D I A V E W E S N |
| | | G Q P E N N Y K T T P P V L D S D |
| | | G S F F L Y S K L T V D K S R W Q |
| | | Q G N V F S C S V M H E A L H N H |
| | | Y T Q K S L S L S P G K |
| 25 | Heavy chain | E V Q L V E S G G G L V Q P G R S |
| | amino acid | L R L S C A A S G F T F D D Y A M |
| | sequence of | H W V R Q A P G K G L E W V S A I |
| | anti-TNF IgG1 | T W N S G H I D Y A D S V E G R F |
| | antibody | T I S R D N A K N S L Y L Q M N S |
| | comprising | L R A E D T A V Y Y C A K V S Y L |
| | mutations at | S T A S S L D Y W G Q G T L V T V |
| | positions | S S A S T K G P S V F P L A P S S |
| | F243A and | K S T S G G T A A L G C L V K D Y |
| | V264A | F P E P V T V S W N S G A L T S G |
| | | V H T F P A V L Q S S G L Y S L S |
| | | S V V T V P S S S L G T Q T Y I C |
| | | N V N H K P S N T K V D K K V E P |
| | | K S C D K T H T C P P C P A P E L |
| | | L G G P S V F L A P P K P K D T L |
| | | M I S R T P E V T C V V A D V S H |
| | | E D P E V K F N W Y V D G V E V H |
| | | N A K T K P R E E Q Y N S T Y R V |
| | | V S V L T V L H Q D W L N G K E Y |
| | | K C K V S N K A L P A P I E K T I |
| | | S K A K G Q P R E P Q V Y T L P P |
| | | S R D E L T K N Q V S L T C L V K |
| | | G F Y P S D I A V E W E S N G Q P |
| | | E N N Y K T T P P V L D S D G S F |
| | | F L Y S K L T V D K S R W Q Q G N |
| | | V F S C S V M H E A L H N H Y T Q |
| | | K S L S L S P G K |
| 26 | Heavy chain | E V Q L V E S G G G L V Q P |
| | amino acid | G R S L R L S C A A S G F T |
| | sequence of | F D D Y A M H W V R Q A P G |
| | anti-TNF IgG1 | K G L E W V S A I T W N S G |
| | antibody | H I D Y A D S V E G R F T I |
| | comprising | S R D N A K N S L Y L Q M N |
| | mutations at | S L R A E D T A V Y Y C A K |
| | positions | V S Y L S T A S S L D Y W G |
| | M252Y, S254T, | Q G T L V T V S S A S T K G |
| | T256E, H433K, | P S V F P L A P S S K S T S |
| | N434F | G G T A A L G C L V K D Y F |
| | | P E P V T V S W N S G A L T |
| | | S G V H T F P A V L Q S S G |
| | | L Y S L S S V V T V P S S S |
| | | L G T Q T Y I C N V N H K P |
| | | S N T K V D K K V E P K S C |
| | | D K T H T C P P C P A P E L |
| | | L G G P S V F L F P P K P K |
| | | D T L Y I T R E P E V T C V |
| | | V V D V S H E D P E V K F N |
| | | W Y V D G V E V H N A K T K |
| | | P R E E Q Y N S T Y R V V S |
| | | V L T V L H Q D W L N G K E |
| | | Y K C K V S N K A L P A P I |
| | | E K T I S K A K G Q P R E P |
| | | Q V Y T L P P S R D E L T K |
| | | N Q V S L T C L V K G F Y P |
| | | S D I A V E W E S N G Q P E |
| | | N N Y K T T P P V L D S D G |
| | | S F F L Y S K L T V D K S R |
| | | W Q Q G N V F S C S V M H E |
| | | A L K F H Y T Q K S L S L S |
| | | P G K |
| 27 | Fc region | T C P P C P A P E L L G G P S V F |
| | (wildtype) | L F P P K P K D T L M I S R T P E |
| | | V T C V V V D V S H E D P E V K F |
| | | N W Y V D G V E V H N A K T K P R |
| | | E E Q Y N S T Y R V V S V L T V L |
| | | H Q D W L N G K E Y K C K V S N K |
| | | A L P A P I E K T I S K A K G Q P |
| | | R E P Q V Y T L P P S R D E L T K |
| | | N Q V S L T C L V K G F Y P S D I |
| | | A V E W E S N G Q P E N N Y K T T |
| | | P P V L D S D G S F F L Y S K L T |
| | | V D K S R W Q Q G N V F S C S V M |
| | | H E A L H N H Y T Q K S L S L S P |
| | | G |
| 28 | Fc region | A E P K S C D K T H T C P P C P A |
| | (wildtype) | P E L L G G P S V F L F P P K P K |
| | | D T L M I S R T P E V T C V V V D |
| | | V S H E D P E V K F N W Y V D G V |
| | | E V H N A K T K P R E E Q Y N S T |
| | | Y R V V S V L T V L H Q D W L N G |
| | | K E Y K C K V S N K A L P A P I E |
| | | K T I S K A K G Q P R E P Q V Y T |
| | | L P P S R D E L T K N Q V S L T C |
| | | L V K G F Y P S D I A V E W E S N |
| | | G Q P E N N Y K T T P P V L D S D |
| | | G S F F L Y S K L T V D K S R W Q |
| | | Q G N V F S C S V M H E A L H N H |
| | | Y T Q K S L S L S P G |

1) An Fc-containing polypeptide comprising mutations at amino acid positions 252, 254, 256, 433, 434, 243 and 264 of the Fc region, wherein the numbering is according to the EU index as in Kabat, wherein the Fc-containing polypeptide comprises N-glycans, and wherein at least 30%, 40%, 50%, 60%, 70%, 80% or 90% of the N-glycans on the Fc-containing polypeptide comprise an N-linked oligosaccharide structure selected from the group consisting of SA₍₁₋₄₎Gal₍₁₋₄₎GlcNAc₍₂₋₄₎Man₃GlcNAc₂.

2) The Fc-containing polypeptide of claim 1, wherein the sialic acid residues in the sialylated N-glycans are attached via α-2,6 linkages.

3) The Fc-containing polypeptide of any one of claims 2 wherein the Fc-containing polypeptide is an antibody or an antibody fragment.

4) The Fc-containing polypeptide of any one of claims 2, wherein the Fc-containing polypeptide has increased FcRn binding and has one or more of the following properties when compared to a parent Fc-containing polypeptide:
a) reduced effector function,
b) increased anti-inflammatory properties,
c) increased sialylation,
d) increased bioavailability when administered parenterally,
e) reduced binding to FcγRI, FcγRIIa and FcγRIIIa,
f) increased binding to FcγRIIb; and
g) increased affinity to human FcRn at pH6 and pH7.

5) The Fc-containing polypeptide of claim 1, wherein the mutations at position 252, 254, 256, 433 and 434 are: M252Y, S254T, T256E, H433K and N434F.

6) The Fc-containing polypeptide of claim 5, wherein the mutations at positions 243 and 264 are selected from the group consisting of:
a) F243A and V264A;
b) F243Y and V264G;
c) F243T and V264G;
d) F243L and V264A;
e) F243L and V264N; and
f) F243V and V264G.

7) The Fc-containing polypeptide of claim 1, wherein the mutations are: M252Y, S254T, T256E, H433K, N434F, F243A and V264A.

8) A method for producing a Fc-containing polypeptide in a host cell comprising:
a) providing a genetically modified host cell that has been genetically engineered to produce an Fc-containing polypeptide comprising sialylated N-glycans, wherein the host cell comprises a nucleic acid encoding mutations at amino acid positions 252, 254, 256, 433, 434, 243 and 264 of the Fc region, wherein the numbering is according to the EU index as in Kabat;
b) culturing the host cell under conditions which cause expression of the Fc-containing polypeptide; and
c) isolating the Fc-containing polypeptide from the host cell,
wherein the Fc-containing polypeptide is an antibody or an antibody fragment, and wherein at least 30%, 40%, 50%, 60%, 70%, 80% or 90% of the N-glycans on the Fc-containing polypeptide comprise an N-linked oligosaccharide structure selected from the group consisting of SA₍₁₋₄₎Gal₍₁₋₄₎GlcNAc₍₂₋₄₎Man₃GlcNAc₂.

9) The method of claim 8, wherein the sialic acid residues in the sialylated N-glycans are attached via α-2,6 linkages.

10) (canceled)

11) (canceled)

12) (canceled)

13) (canceled)

14) (canceled)

15) The method of claim 8, wherein the mutations are: M252Y, S254T, T256E, H433K, N434F, F243A and V264A.

16) A method of increasing the anti-inflammatory properties or decreasing cytotoxicity of an Fc-containing polypeptide comprising introducing mutations at positions 252, 254, 256, 433, 434, 243 and 264 of the Fc region, wherein the numbering is according to the EU index as in Kabat;
wherein the Fc-containing polypeptide has improved FcRn binding and increased anti-inflammatory properties or decreased cytotoxicity when compared to a parent Fc-containing polypeptide.

17) (canceled)

18) (canceled)

19) The method of claim 16, wherein the mutations are: M252Y, S254T, T256E, H433K, N434F, F243A and V264A.

20) The method of claim 16, wherein the Fc-containing polypeptide is an antibody or an antibody fragment, and wherein at least 30%, 40%, 50%, 60%, 70%, 80% or 90% of the N-glycans on the Fc-containing polypeptide comprise an N-linked oligosaccharide structure selected from the group consisting of SA₍₁₋₄₎Gal₍₁₋₄₎GlcNAc₍₂₋₄₎Man₃GlcNAc₂.

21) A method of treating an inflammatory condition in a subject in need thereof comprising: administering to the subject a therapeutically effective amount of an Fc-containing polypeptide comprising mutations at positions 252, 254, 256, 433, 434, 243 and 264 of the Fc region, wherein the numbering is according to the EU index as in Kabat.

22) (canceled)

23) (canceled)

24) The method of claims 22, wherein the mutations are: M252Y, S254T, T256E, H433K, N434F, F243A and V264A.

25) The method of claims 22, wherein the Fc-containing polypeptide is an antibody or an antibody fragment, and wherein at least 30%, 40%, 50%, 60%, 70%, 80% or 90% of the N-glycans on the Fc-containing polypeptide comprise an N-linked oligosaccharide structure selected from the group consisting of SA₍₁₋₄₎Gal₍₁₋₄₎GlcNAc₍₂₋₄₎Man₃GlcNAc₂.
